(19) United States
(12) Patent Application Publication
(10) Pub. No.: US 2023/0295637 A1
(43) Pub. Date: Sep. 21, 2023

(54) METHODS FOR REDUCING RNA IMMUNOGENICITY AND RNA MOLECULES WITH DECREASED IMMUNOGENICITY

(71) Applicant: RiboPro B.V., Oss (NL)

(72) Inventors: Alexander Henrik BARON VAN ASBECK, Nijmegen (NL); Jürgen Wilhelmus Carolus DIEKER, Zutphen (NL)

(73) Assignee: RiboPro B.V., Oss (NL)

(21) Appl. No.: 17/922,058

(22) PCT Filed: Apr. 30, 2021
(86) PCT No.: PCT/NL2021/050284

§ 371 Date: Oct. 28, 2022

(30) Foreign Application Priority Data
Apr. 30, 2020 (NL) 2025475

(51) Int. Cl. C12N 15/52; C12N 15/10

(52) U.S. Cl. CPC C12N 15/52; C12N 15/102; C12N 2310/20; C12N 2310/334

(57) ABSTRACT

The present invention relates to a method for decreasing the immunogenicity of an RNA molecule and/or at least maintaining the translation efficacy thereof. The present invention further relates to an RNA molecule, which is modified as compared to a corresponding wildtype RNA molecule, wherein the exchange of codons results in the total cytidine content of the modified RNA molecule being at least 10% less than the total cytidine content of the corresponding RNA molecule transcribed from said wildtype DNA sequence. The present invention also relates to an RNA molecule, wherein the exchange of codons results in the total uridine content of the modified RNA molecule being at least 10% less than the total uridine content of the corresponding RNA molecule transcribed from said wild-type DNA sequence. Finally, the present invention relates to the use of an RNA molecule of this invention in genome editing.

This application is a national stage filing under 35 U.S.C. 371 of pending International Application No. PCT/NL2021/050284, filed Apr. 30, 2021, which claims priority to Netherlands Patent Application No. 2025475, filed Apr. 30, 2020, the entirety of which applications are incorporated by reference herein.

### SEQUENCE LISTING

The instant application contains an electronic Sequence Listing which has been submitted electronically in ASCII format and is hereby incorporated by reference in its entirety. Said ASCII copy, created on Mar. 17, 2023, is named 1865_00285_ReplacmentSeqeunceListing.txt and is 30,784 bytes in size.

The present invention relates to methods for reducing the immunogenicity of RNA molecules and to RNA molecules thus obtained having decreased immunogenicity. The invention further relates to such RNA molecules for use in therapy and diagnosis.

RNA has, since its discovery, been increasingly recognized to play a critical role in the biology of virtually every form of life. RNA molecules have been shown to be involved in the relay of genetic information, catalyzing biological reactions, as well as sensing, communicating and responding to cellular signaling, among other functions. As such, the interest in RNA has been steadily increasing, with a corresponding increase in the manipulation of RNA and the use of RNA as research tool. More recently, advances in the synthesis, purification and delivery of RNA into compartments of cells have led to novel applications of RNA, including the use in therapy.

Within this latter category, the use of in vitro-transcribed long RNA molecules (IVT RNA), such as (poly)peptide-encoding messenger RNA and long non-coding RNA, is emerging as a new type of gene therapy. However, immunological activation by the exogenous supplied RNA has limited the uptake of IVT RNA as therapy due to unacceptable side effects related to innate immunity and limited efficacy. Efficacy of messenger RNA is related to the amount of protein translation from a given mRNA dose and is negatively affected by intracellular innate immunogenicity of exogenous supplied RNA. IVT mRNA is similar to endogenous mRNA, but differs in chemical modifications and the position of such modifications. In addition, the trafficking into the inside of the cell exposes IVT RNA to innate immunity sensors typically not or less encountered by endogenous RNA. Upon detection by such innate immunity sensors, including TLR3, TLR7, TLR8, RIG-I, OAS and MDAS, pro-inflammatory cytokines are released and protein translation is inhibited. Cellular toxicity occurs due to protein synthesis inhibition and local/systemic toxicity due to cytokine release. The efficacy of the supplied IVT RNA consequently decreased dramatically as it is no longer translated. It is therefore of great importance that the innate immunity is avoided.

In various prior art documents, it is disclosed that particular nucleosides are replaced by modified nucleosides in order to prevent activation of the innate immunity. Karikó, K. et al. (2005) studied the exchange of uridine for pseudouridine in order to make RNA molecules less immunogenic, while remaining translatable. Several related approaches have shown the incorporation of other chemically modified nucleosides in IVT RNA to reduce immune activation via cytoplasmic and endocytotic innate immune sensors. Chemical modifications of other nucleosides would result in an equal or better reduction in immunogenicity. For example, in US2012/0195936 A1, it is shown that the chemically modified nucleoside m6A would display the lowest cytokine release and outperform other nucleoside modifications. These findings are, however, at odds with the findings of Karikó et al. (US 2019/0153428) where the non-modified adenosine is described as a less immunogenic nucleoside compared to the non-modified Uridine nucleoside it replaces.

Although some of the chemically modified nucleosides are naturally occurring, their application in IVT RNA is not without problems. In the mammalian cell, for instance, the chemical modification of nucleotides is a post-transcriptional process that is highly regulated and position specific. However, in many approaches, all of the instances of a particular nucleoside are replaced with a chemically modified nucleoside. Even when partial replacement (e.g. 25%) is practiced, the amount of modifications often exceeds natural levels and the positioning of the modified nucleoside may be different. This is relevant, because chemically modified nucleosides have been shown to alter the binding properties of corresponding tRNAs during protein translation and the formation of secondary structures. As one example, the naturally occurring pseudouridine modification, has been shown to cause read-through of the stop codon, introducing novel, and thus potentially auto-immunity inducing or toxic, (poly)peptides. Obviously, the induction of novel, non-endogenous peptides is to be avoided in any therapy setting.

As an alternative approach to the use of modified nucleosides, sequence-engineering was applied to reduce the immunogenicity of RNA and enhance the therapeutic efficacy (Thess, A., et al. (2015)). As part of this method, the coding sequence of the mRNA was enriched with GC-rich codons and the resultant sequence was reported to be less immunogenic and showing higher translation. In a similar fashion, Karikó et al. reported reduced immunogenicity, and corresponding increases in translation, from mRNA, whereof the Uridine content was reduced by replacement with other nucleosides, preferably adenosine.

However, strongly biased nucleoside usage can provide problems during synthesis of the DNA template and/or cause single strand RNA, including messenger RNA, to fold back onto itself via its exposed nucleoside and form (too) strong secondary structures, reducing translation and inducing premature translation termination. Especially, sequences with a very high or low GC-content present problems during DNA synthesis.

Although aforementioned studies have shown a reduction in immunogenicity and consequently enhanced translation of the mRNA sequence, additional improvement is required because a further reduction of the RNA induced immune response enlarges the therapeutic window by increasing the safety and increasing the protein yield per mRNA dose. This is needed to be able to use higher RNA doses in therapy and achieve protein levels suitable for protein replacement therapies requiring very high protein amounts or to achieve sufficient levels of very unstable proteins. Also, high RNA doses could be applied in RNA therapy in diseases with high inflammatory activity, in which additional cytokine release would be extra detrimental.
It has been surprisingly found that a higher decrease in immunogenicity can be achieved as compared to the approaches used in the prior art by reducing the cytidine content of RNA. Reducing the cytidine content of an RNA molecule results in the same protein being produced with the same fidelity. It was furthermore found that particular good results are obtained when the cytidines are replaced by other non-modified nucleotides. Preferably, such non-modified nucleotides are chosen such that the corresponding codon encodes the same or a similar type of amino acid.

The invention therefore relates to a method for decreasing the immunogenicity of an RNA molecule and/or at least maintaining the translation efficacy thereof, which method comprises the steps of:
a) providing a wildtype DNA sequence as a template for RNA transcription;
b) selecting from the DNA sequence the coding sequence, which comprises the sequence from the ATG codon to the first in-frame stop codon;
c) dividing the coding sequence into codons;
d) exchanging one or more codons that comprise one or more cytidine nucleotides for an available alternative codon comprising less cytidine nucleotides and resulting in the same or similar amino acid to obtain a DNA molecule with a modified DNA sequence; and
e) producing a modified RNA molecule from the DNA molecule with the modified DNA sequence,

wherein the exchange of codons results in the total cytidine content of the modified RNA molecule being at least 10% less than the total cytidine content of the corresponding RNA molecule transcribed from said wild-type DNA sequence.

In a further embodiment, the method comprises the additional step of repeating step d) with codons comprising thymidine nucleotides before producing the modified RNA molecule, wherein the exchange of codons results in the total uridine content of the modified RNA molecule being at least 10% less than the total uridine content of the corresponding RNA molecule transcribed from said wild-type DNA sequence.

The method of the invention thus results in an RNA molecule, which is modified as compared to a corresponding wildtype RNA molecule, wherein the modification comprises a reduction of cytidine nucleotides to the extent that at least 10% of the cytidine nucleotides present in the RNA sequence of the wildtype RNA molecule are replaced by nucleotides other than cytidine in the modified RNA molecule or deleted. It was found that with a modified RNA molecule having a reduced cytidine content compared to the corresponding non-modified, wildtype RNA molecule also a higher protein translation is achieved than with the corresponding non-modified, wildtype RNA. Modified mRNA molecules having a reduced cytidine content according to the invention are also called C-depleted mRNA molecules.

Decreased immunogenicity and higher protein translation is also achieved by the combined reduction of the uridine and cytidine content. In a further embodiment, the invention thus relates to an RNA molecule which is modified as compared to a corresponding wildtype RNA molecule, wherein the modification further comprises a reduction of uridine nucleotides to the extent that at least 10% of the uridine nucleotides present in the RNA sequence of the wildtype RNA molecule are nucleotides other than uridine in the modified RNA molecule or deleted. Modified mRNA molecules optionally having a reduced uridine content according to the invention are also called herein C- and optionally U-depleted mRNA molecules.

When both cytidines and uridines are reduced the mRNA molecules are called UC-depleted or CU-depleted. When only the U-content is decreased the molecules are called U-depleted mRNA molecules.

Preferably, in order of increased preference at least 10, 15, 20, 25, 30, 35, 40, 45, 50% of the cytidine and optionally uridine nucleotides of the RNA sequence of the wildtype RNA molecule are replaced by a nucleotide that is not cytidine or uridine, respectively, or deleted. This means that a molecule can have any combination of percentages replacement for C and U between and 50%. For example, an mRNA molecule can have 20% less cytidine nucleotides and 10% less uridine nucleotides as compared to the corresponding wild type sequences, or any other combination.
Preferably, the nucleotides replacing the cytidines or uridines of the wild type RNA molecule in the modified RNA molecule are primary nucleotides, i.e. nucleotides that are not modified and comprise one of the five nucleobases adenine (A), cytosine (C), guanine (G), thymine (T), and uracil (U), in particular adenine and guanine. A, C, G, T and U are also called the canonical nucleotides.
The RNA molecule of the invention can for example be a long non-coding RNA molecule. Long non-coding RNAs (also known as long ncRNAs, lncRNA) are a type of RNA transcripts with lengths exceeding 200 nucleotides that are not translated into protein. Long ncRNAs can have a myriad of functions, for example in the regulation of gene transcription. These functions are mostly related to the RNA nucleotide sequence (e.g. by binding to other types of RNA) or to the secondary structure (e.g. binding to intracellular proteins). Decreasing the immunogenicity of long non-coding RNAs enables improvement of these functions.
In another embodiment, the RNA molecule of the invention is an mRNA molecule. mRNA molecules function as a template for polypeptide or protein synthesis. Decreased immunogenicity and the resulting enhanced expression of these molecules provide that mRNA molecules are longer present in higher concentrations at targeted positions. Since the translation of the mRNA molecule is also enhanced, therapies including replacement of defective or absent protein are therefore improved using the mRNA molecules of this invention.

The RNA molecule of the invention is modified as compared to a wildtype RNA molecule by deleting and/or substituting one or more of the cytidine nucleotides and optionally one or more of the uridine nucleotides. In the coding sequence of an mRNA, it is usually not possible to delete single nucleotides or two consecutive nucleotides as this would result in a frameshift. It is possible to delete an entire codon but this leads to a deletion of an amino acid. It is preferred to replace the cytidine and/or uridine with another non-modified nucleotide that leaves the corresponding amino acid intact. Due to the degeneracy of the genetic code, there is usually a choice from more than one alternative nucleotide.

Deletion or substitution can be performed by replacing or deleting nucleotides from existing RNA molecules, for example by gene-editing techniques, or providing newly synthesized RNA molecules having a modified sequence as compared to a wildtype RNA molecule.
In long non-coding RNAs individual cytidines or uridines and even stretches thereof can be deleted without adverse effect.
A large subgroup of RNA molecules are mRNA molecules encoding for one or more amino acids which form polypeptides or proteins. The structure of polypeptides or proteins is defined by their amino acid sequence and nucleotide modification of mRNA molecules can thus potentially affect the structure of polypeptides or proteins. However, multiple nucleotide combinations can result in the same amino acid. It is therefore possible to modify an mRNA molecule without affecting the amino acids sequence it encodes.
In one embodiment of the invention, the modified RNA molecule is an mRNA and the amino acid sequence of the polypeptide or protein encoded by the modified mRNA molecule is the same as the amino acid sequence of the polypeptide or protein encoded by the wildtype mRNA molecule. This is achieved by replacing wildtype codons with codons encoding the same amino acid but having no or less cytidine and optionally uridine nucleotides.
In another embodiment, the modified RNA molecule is an mRNA and the amino acid sequence of the polypeptide or protein encoded by the modified mRNA molecule is different from the amino acid sequence of the polypeptide or protein encoded by the wildtype mRNA molecule. Usually, it is preferred to keep the encoded polypeptide or protein unchanged to avoid affecting its function. In some situations, it may not be possible to exchange sufficient codons to achieve the desired reduction. Then, it is preferred to include one or more conservative substitutions. Conservative substitutions are least likely to influence the three-dimensional structure of the polypeptide or protein and consequently its biological function.
Preferably, the difference between the amino acid sequence encoded by the modified RNA sequence compared to the amino acid sequence encoded by the wildtype RNA sequence is kept as low as possible and is less than 1/200 codons, preferably less than 1/1000 codons, more preferably less than 1/5000 codons, even more preferably 1/10000 codons, most preferably 1/50000 codons.
The RNA sequence is preferably modified by substituting cytidine and optionally uridine nucleotides by adenosine or guanosine nucleotides. When only cytidine depletion is used, cytidines can also be replaced by uridines. Each cytidine in a modified RNA molecule can be replaced by either an adenosine or a guanosine nucleotide. Each uridine in a modified RNA molecule can be replaced by either an adenosine or a guanosine nucleotide.
In one embodiment, the RNA molecule is an mRNA and the cytidine content and optionally the uridine content is reduced in the coding region of the mRNA.
In another embodiment, the RNA molecule is an mRNA and the cytidine content and optionally the uridine content is reduced in the non-coding region of the mRNA, in particular in the 5′UTR region and/or 3′UTR region, the 5′ cap and poly-A tail. It should, however, be avoided that these modifications are detrimental to the translation of the mRNA. In some embodiments, modification of the non-coding regions can also lead to an improvement of translation.
Translatability may depend on the system of application. According to the invention it is shown that the claimed method results in enhanced translation in human cells in vitro, in mouse cells in vitro and in mice in vivo. Although the effect of C depletion and optionally U depletion on translatability is always the same or better and the relative relationship between sequence modifications (for example C-depleted versus UC-depleted) is always at least the same, the relative magnitude of the enhancement in protein translation may be variable per system and sequence. However, given the evolutionary conservation of ribosome function and innate immune system (TLRs, MDA5, OAs2, RIG-I) it stands to reason that the observed effects are applicable to a wide range of species.
Alternatively, instead of modifying the non-coding regions, natural or synthetic UTRs with an as low as possible C-usage or UC-usage can be chosen.
In a further embodiment, the cytidine content and optionally the uridine content is reduced both in the coding region of the mRNA and in the non-coding region of the mRNA, in particular in the 5 ′UTR region and/or 3′UTR region.
The present invention is applicable to all known human or animal RNA molecules. The wildtype sequences of RNA molecules that can be modified according to the invention to produce modified RNA molecules that are less immunogenic and have the same or better translatability can be found in the NCBI database.
Http://www.ensembl.org/info/data/ftp/index.html is a general database containing all nucleotide sequences of interest. The specific database reference for a number of species is found in the table below.

| | |
|---|---|
| 1. Homo sapiens (human) | http://ftp.ensembl.org/pub/release- |
| | 103/fasta/homo_sapiens/cdna/ |
| | Homo_sapiens.GRCh38.cdna.all.fa.gz |
| 2. Mus musculus (mouse) | http://ftp.ensembl.org/pub/release- |
| | 103/fasta/mus_musculus/cdna/ |
| | Mus_musculus.GRCm39.cdna.all.fa.gz |
| 3. Danio Rero (zebrafish) | http://ftp.ensembl.org/pub/release- |
| | 103/fasta/danio_rerio/cdna/ |
| | Danio_rerio.GRCz11.cdna.all.fa.gz |
| 4. Vicugna pacos (alpaca) | http://ftp.ensembl.org/pub/release- |
| | 103/fasta/mus_spretus/cdna/ |
| | Mus_spretus.SPRET_EiJ_v1.cdna.all.fa.gz |
| 5. Marmota marmota (marmot) | http://ftp.ensembl.org/pub/release- |
| | 103/fasta/marmota_marmota_marmota/cdna/ |
| | Marmota_marmota_marmota.marMar2.1.cdna.all.fa.gz |
| 6. Bison bison (bison) | http://ftp.ensembl.org/pub/release- |
| | 103/fasta/bison_bison_bison/cdna/ |
| | Bison_bison_bison.Bison_UMD1.0.cdna.all.fa.gz |
| 7. Camelis dromedarius (Arabian | http://ftp.ensembl.org/pub/release- |
| camel) | 103/fasta/camelus_dromedarius/cdna/ |
| | Camelus_dromedarius.CamDro2.cdna.all.fa.gz |
| 8. Dasypus novemcinctus | http://ftp.ensembl.org/pub/release- |
| (armadillo) | 103/fasta/dasypus_novemcinctus/cdna/ |
| | Daypus_novemcinctus.Dasnov3.0.cdna.all.fa.gz |
| 9. Gadus morhua (cod) | http://ftp.ensembl.org/pub/release- |
| | 103/fasta/gadus_morhua/cdna/ |
| | Gadus_morhua.gadMor3.0.cdna.all.fa.gz |
| 10. Clupea harengus (herring) | http://ftp.ensembl.org/pub/release- |
| | 103/fasta/clupea_harengus/cdna/ |
| | Clupea_harengus.Ch_v2.0.2.cdna.all.fa.gz |
| 11. Salmo salar (salmon) | http://ftp.ensembl.org/pub/release- |
| | 103/fasta/salmo_salar/cdna/ |
| | Salmo_salar.ICSASG_v2.cdna.all.fa.gz |
| 12. Oreochromis aureus (tilapia) | http://ftp.ensembl.org/pub/release- |
| | 103/fasta/oreochromis_aureus/cdna/ |
| | Oreochromis_aureus.ASM587006v1.cdna.all.fa.gz |
| 13. Pan paniscus (bonobo) | http://ftp.ensembl.org/pub/release- |
| | 103/fasta/pan_paniscus/cdna/ |
| | Pan_paniscus.panpan1.1.cdna.all.fa.gz |
| 14. Cavia aperea (Brazilian guinea | http://ftp.ensembl.org/pub/release- |
| pig) | 103/fasta/cavia_aperea/cdna/ |
| | Cavia_aperea.CavAp1.0.cdna.all.fa.gz |
| 15. Salmo trutta (trout) | http://ftp.ensembl.org/pub/release- |
| | 103/fasta/salmo_trutta/cdna/ |
| | Salmo_trutta.fSalTru1.1.cdna.all.fa.gz |
| 16. Otolemur garnettii (northern | http://ftp.ensembl.org/pub/release- |
| greater galago) | 103/fasta/otolemur_garnetti/cdna/ |
| | Otolemur_garnetti.OtoGar3.cdna.all.fa.gz |
| 17. Ciona intestinalis (vase | http://ftp.ensembl.org/pub/release- |
| tunicate) | 103/fasta/ciona_intestinalis/cdna/ |
| | Ciona_intestinalis.KH.cdna.all.fa.gz |
| 18. Ciona savignyi | http://ftp.ensembl.org/pub/release- |
| | 103/fasta/ciona_savignyi/cdna/ |
| | Ciona_savignyi.CSAV2.0.cdna.all.fa.gz |
| 19. Caenorhabditis elegans | http://ftp.ensembl.org/pub/release- |
| | 103/fasta/caenorhabditis_elegans/cdna/ |
| | Caenorhabditis_elegans.WBcel235.cdna.all.fa.gz |
| 20. Cebus capucinus imitator | http://ftp.ensembl.org/pub/release- |
| (capuchin) | 103/fasta/cebus_capucinus/cdna/ |
| | Cebus_capucinus.Cebus_imitator-1.0.cdna.all.fa.gz |
| 21. Felis catus (cat) | http://ftp.ensembl.org/pub/release- |
| | 103/fasta/felis_catus/cdna/ |
| | Felis_catus.Felis_catus_9.0.cdna.all.fa.gz |
| 22. Ictalurus punctatus (channel | http://ftp.ensembl.org/pub/release- |
| catfish) | 103/fasta/ictalurus_punctatus/cdna/ |
| | Ictalurus_punctatus.IpCoco_1.2.cdna.all.fa.gz |
| 23. Gallus gallus (red junglefowl) | http://ftp.ensembl.org/pub/release- |
| | 103/fasta/gallus_gallus/cdna/ |
| | Gallus_gallus.GRCg6a.cdna.all.fa.gz |
| 24. Pan troglodytes (chimpanzee) | http://ftp.ensembl.org/pub/release- |
| | 103/fasta/pan_troglodytes/cdna/ |
| | Pan_troglodytes.Pan_tro_3.0.cdna.all.fa.gz |
| 25. Cricetulus griseus (Chinese | http://ftp.ensembl.org/pub/release- |
| hamster CHOK1GS) | 103/fasta/cricetulus_griseus_chok1gshd/cdna/ |
| | Cricetulus_grisous_chok1gshd.CHOK1GS_HDv1.cdna.all.fa.gz |
| 26. Cricetulus griseus (Chinese | http://ftp.ensembl.org/pub/release- |
| hamster CriGri) | 103/fasta/cricetulus_griseus_crigri/cdna/ |
| | Cricetulus_griseuscrigri.CriGri_1.0.cdna.all.fa.gz |
| 27. Cricetulus griseus (Chinese | http://ftp.ensembl.org/pub/release- |
| hamster PICR) | 103/fasta/cricetulus_griseus_picr/cdna/ |
| | Cricetulus_griseus_picr.CriGri-PICR.cdna.all.fa.gz |
| 28. Oncorhynchus tshawytscha | http://ftp.ensembl.org/pub/release- |
| (salmon) | 103/fasta/oncorhynchus_tshawytscha/cdna/ |
| | Oncorhynchus_tshawytscha.Otsh_v1.O.cdna.all.fa.gz |
| 29. Latimeria chalumnae | http://ftp.ensembl.org/pub/release- |
| (coelacanth) | 103/fasta/latimeria_chalumnae/cdna/ |
| | Latimeria_chalumnae.LatCha1.cdna.all.fa.gz |
| 30. Oncorhynchus kisutch (salmon) | http://ftp.ensembl.org/pub/release- |
| | 103/fasta/oncorhynchus_kisutch/cdna/ |
| | Oncorhynchus_kisutch.Okis_V2.cdna.all.fa.gz |
| 31. Serinus canaria (common | http://ftp.ensembl.org/pub/release- |
| canary) | 103/fasta/serinus_canaria/cdna/ |
| | Serinus_canaria.SCA1.cdna.all.fa.gz |
| 32. Cyprinus carpio (common | http://ftp.ensembl.org/pub/release- |
| carp) | 103/fasta/cyprinus_carpio/cdna/ |
| | Cyprinus_carpio.common_carp_genome.cdna.all.fa.gZ |
| 33. Bos taurus (cow) | http://ftp.ensembl.org/pub/release- |
| | 103/fasta/bos_taurus/cdna/ |
| | Bos_taurus.ARS-UCD1.2.cdna.all.fa.gz |
| 34. Macaca fascicularis (crab- | http://ftp.ensembl.org/pub/release- |
| eating macaque) | 103/fasta/macaca_fascicularis/cdna/ |
| | Macaca_fascicularis.Macaca_fascicularis_6.0.cdna.all.fa.gz |
| 35. Denticeps clupeoides (herring) | http://ftp.ensembl.org/pub/release- |
| | 103/fasta/denticeps_clupeoides/cdna/ |
| | Denticeps_clupeoides.fDenClu1.1.cdna.all.fa.gz |
| 36. Canis lupus familiaris (dog) | http://ftp.ensembl.org/pub/release- |
| | 103/fasta/canis_lupus_familiaris/cdna/ |
| | Canis_lupus_familiaris.CanFam3.1.cdna.all.fa.gz |
| 37. Bos grunniens (domestic yak) | http://ftp.ensembl.org/pub/release- |
| | 103/fasta/bos_grunniens/cdna/ |
| | Bos_grunniens.LU_Bosgni_v3.0.cdna.all.fa.gz |
| 38. Equus asinus asinus (donkey) | http://ftp.ensembl.org/pub/release- |
| | 103/fasta/equus_asinus/cdna/ |
| | Equus_asinus_asinus.ASM303372v1.cdna.all.fa.gz |
| 39. Drosophila melanogaster (fruit | http://ftp.ensembl.org/pub/release- |
| fly) | 103/fasta/drosophila_melanogaster/cdna/ |
| | Drosophila_melanogaster.BDGP6.32.cdna.all.fa.gz |
| 40. Anas platyrhynchos | http://ftp.ensembl.org/pub/release- |
| platyrhynchos (duck) | 103/fasta/anas_platyrhynchos_platyrhynchos/cdna/ |
| | Anas_platyrhynchos_platyrhynchos.CAU_duck1.0.cdna.all.fa.gz |
| 41. Nomascus leucogenys (gibbon) | http://ftp.ensembl.org/pub/release- |
| | 103/fasta/nomascus_leucogenys/cdna/ |
| | Nomascus_leucogenys.Nleu_3.0.cdna.all.fa.gz |
| 42. Capra hircus (goat) | http://ftp.ensembl.org/pub/release- |
| | 103/fasta/capra_hircus/cdna/ |
| | Capra_hircus.ARS1.cdna.all.fa.gz |
| 43. Mesocricetus auratus (golden | http://ftp.ensembl.org/pub/release- |
| hamster) | 103/fasta/mesocricetus_auratus/cdna/ |
| | Mesocricetus_auratus.MesAur1.0.cdna.all.fa.gz |
| 44. Chrysolophus pictus (pheasant) | http://ftp.ensembl.org/pub/release- |
| | 103/fasta/chrysolophus_pictus/cdna/ |
| | Chrysolophus_pictus.Chrysolophus_pictus_GenomeV1.0.cdna.all.fa.gz |
| 45. Carassius auratus (goldfish) | http://ftp.ensembl.org/pub/release- |
| | 103/fasta/carassius_auratus/cdna/ |
| | Carassius_auratus.ASM336829v1.cdna.all.fa.gz |
| 46. Gorilla gorilla gorilla | http://ftp.ensembl.org/pub/release- |
| | 103/fasta/gorilla_gorilla/cdna/ |
| | Gorilla_gorilla.gorGor4.cdna.all.fa.gz |
| 47. Rhinolophus ferrumequinum | http://ftp.ensembl.org/pub/release- |
| (bat) | 103/fasta/rhinolophus_ferrumequinum/cdna/ |
| | Rhinolophus_ferrumequinum.mRhiFer1_v1.p.cdna.all.fa.gz |
| 48. Cavia porcellus (cavia) | http://ftp.ensembl.org/pub/release- |
| | 103/fasta/cavia_porcellus/cdna/ |
| | Cavia_porcellus.Cavpor3.0.cdna.all.fa.gz |
| 49. Equus caballus (horse) | http://ftp.ensembl.org/pub/release- |
| | 103/fasta/equus_caballus/cdna/ |
| | Equus_caballus.EquCab3.0.cdna.all.fa.gz |
| 50. Macaca mulatta (rhesus | http://ftp.ensembl.org/pub/release- |
| macaque) | 103/fasta/macaca_mulatta/cdna/ |
| | Macaca_mulatta.Mmul_10.cdna.all.fa.gz |
| 51. Pteropus vampyrus (large | http://ftp.ensembl.org/pub/release- |
| flying fox) | 103/fasta/pteropus_vampyrus/cdna/ |
| | Pteropus_vampyrus.pteVam1.cdna.all.fa.gz |
| 52. Myotis lucifugus (little brown | http://ftp.ensembl.org/pub/release- |
| bat) | 103/fasta/myotis_lucifugus/cdna/ |
| | Myotis_lucifugus.Myoluc2.0.cdna.all.fa.gz |
| 53. Mus musculus (house mouse | http://ftp.ensembl.org/pub/release- |
| 129S1/SvlmJ) | 103/fasta/mus_musculus_129slsvimj/cdna/ |
| | Mus_musculus_129slsvimj.129S1_SvImJ_v1.cdna.all.fa.gz |
| 54. Mus musculus (house mouse | http://ftp.ensembl.org/pub/release- |
| A/J) | 103/fasta/mus_musculus_aj/cdna/ |
| | Mus_musculus_aj.A_J_v1.cdna.all.fa.gz |
| 55. Mus musculus (house mouse | http://ftp.ensembl.org/pub/release- |
| AKR/J) | 103/fasta/mus_musculus_akrj/cdna/ |
| | Mus_musculus_akjr.AKRJ_v1.cdna.all.fa.gz |
| 56. Mus musculus (house mouse | http://ftp.ensembl.org/pub/release- |
| BALB/cJ) | 103/fasta/mus_musculus_balbcj/cdna/ |
| | Mus_musculus_balbcj.BALB_cJ_v1.cdna.all.fa.gz |
| 57. Mus musculus (house mouse | http://ftp.ensembl.org/pub/release- |
| C3H/HeJ) | 103/fasta/mus_musculus_c3hhej/cdna/ |
| | Mus_musculus_c3hhej.C3H_HeJ_v1.cdna.all.fa.gz |
| 58. Mus musculus (house mouse | http://ftp.ensembl.org/pub/release- |
| C57BL/6NJ) | 103/fasta/mus_musculus_c57bl6nj/cdna/ |
| | Mus_musculus_c57bl6nj.C57BL_6NJ_v1.cdna.all.fa.gz |
| 59. Mus musculus (house mouse | http://ftp.ensembl.org/pub/release- |
| CAST/EiJ) | 103/fasta/mus_musculus_casteij/cdna/ |
| | Mus_musculus_casteij.CAST_EiJ_v1.cdna.all.fa.gz |
| 60. Mus musculus (house mouse | http://ftp.ensembl.org/pub/release- |
| CBA/J) | 103/fasta/mus_musculus_cbaj/cdna/ |
| | Mus_musculus_cbaj.CBAJ_v1.cdna.all.fa.gz |
| 61. Mus musculus (house mouse | http://ftp.ensembl.org/pub/release- |
| DBA/2J) | 103/fasta/mus_musculus_dba2j/cdna/ |
| | Mus_musculus_dba2j.DBA_2J_v1.cdna.all.fa.gZ |
| 62. Mus musculus (house mouse | http://ftp.ensembl.org/pub/release- |
| FVB/NJ) | 103/fasta/mus_musculus_fvbnj/cdna/ |
| | Mus_musculus_fybnj.FVB_NJ_v1.cdna.all.fa.yz |
| 63. Mus musculus (house mouse | http://ftp.ensembl.org/pub/release- |
| LP/J) | 103/fasta/mus_musculus_lpj/cdna/ |
| | Mus_musculus_lpj.LP_J_v1.cdna.all.fa.gz |
| 64. Microcebus murinus (gray | http://ftp.ensembl.org/pub/release- |
| mouse lemur) | 103/fasta/microcebus_murinus/cdna/ |
| | Microcebus_murinus.Mmur_3.0.cdna.all.fa.gz |
| 65. Mus musculus (house mouse | http://ftp.ensembl.org/pub/release- |
| NOD/ShiLtJ) | 103/fasta/mus_musculus_nodshiltj/cdna/ |
| | Mus_musculus_nodshiltj.NOD_ShiLtJ_v1.cdna.all.fa.gz |
| 66. Mus musculus (house mouse | http://ftp.ensembl.org/pub/release- |
| NZO/HILtJ) | 103/fasta/mus_musculus_nzohlltj/cdna/ |
| | Mus_musculus_nzohlltj.NZO_HlLtJ_v1.cdna.all.fa.gz |
| 67. Mus musculus (house mouse | http://ftp.ensembl.org/pub/release- |
| PWK/PhJ) | 103/fasta/mus_musculus_pwkphj/cdna/ |
| | Mus_musculus_pwkphj.PWK_PhJ_v1.cdna.all.fa.gZ |
| 68. Mus musculus (house mouse | http://ftp.ensembl.org/pub/release- |
| WSB/EiJ) | 103/fasta/mus_musculus_wsbeij/cdna/ |
| | Mus_musculus_wsbeij.WSB_EiJ_v1.cdna.all.fa.gz |
| 69. Heterocephalus glaber (naked | http://ftp.ensembl.org/pub/release- |
| mole rat) | 103/fasta/heterocephalus_glaber_female/cdna/ |
| | Heterocephalus_glaber_female.HetGla_female_1.0.cdna.all.fa.gz |
| | http://ftp.ensembl.org/pub/release- |
| | 103/fasta/heterocephalus_glaber_male/cdna/ |
| | Heterocephalus_glaber_male.HetGla_1.0.cdna.all.fa.gz |
| 70. Oreochromis niloticus (tilapia) | http://ftp.ensembl.org/pub/release- |
| | 103/fasta/oreochromis_niloticus/cdna/ |
| | Oreochromis_niloticus.O_niloticus_UMD_NMBU.cdna.all.fa.gz |
| 71. Pongo abelii (Sumatran | http://ftp.ensembl.org/pub/release- |
| orangutan) | 103/fasta/pongo_abelii/cdna/ |
| | Pongo_abelii.PPYG2.cdna.all.fa.gz |
| 72. Sus scrofa (wild boar) | http://ftp.ensembl.org/pub/release- |
| | 103/fasta/sus_scrofa/cdna/ |
| | Sus_Sscrofa11.1.cdna.all.fa.gz |
| 73. Sus scrofa (wild boar Bamei) | http://ftp.ensembl.org/pub/release- |
| | 103/fasta/sus_scrofa_bamei/cdna/ |
| | Sus_scrofa_bamei.Bamei_pig_v1.cdna.all.fa.gz |
| 74. Sus scrofa (wild boar | http://ftp.ensembl.org/pub/release- |
| Berkshire) | 103/fasta/sus_scrofa_berkshire/cdna/ |
| | Sus_scrofa_berkshire.Berkshire_pig_v1.cdna.all.fa.gz |
| 75. Sus scrofa (wild boar | http://ftp.ensembl.org/pub/release- |
| Hampshire) | 103/fasta/sus_scrofa_hampshire/cdna/ |
| | Sus_scrofa_hampshire.Hampshire_pig_v1.cdna.all.fa.gz |
| 76. Sus scrofa (wild boar Jinhua) | http://ftp.ensembl.org/pub/release- |
| | 103/fasta/sus_scrofa_jinhua/cdna/ |
| | Sus_scrofa_jinhua.Jinhua_pig_v1.cdna.all.fa.gz |
| 77. Sus scrofa (wild boar Landrace) | http://ftp.ensembl.org/pub/release- |
| | 103/fasta/sus_scrofa_landrace/cdna/ |
| | Sus_scrofa_landrace.Landrace_pig_v1.cdna.all.fa.gz |
| 78. Sus scrofa (wild boar | http://ftp.ensembl.org/pub/release- |
| Largewhite) | 103/fasta/sus_scrofa_largewhite/cdna/ |
| | Sus_scrofa_largewhite.Large_White_v1.cdna.all.fa.gz |
| 79. Sus scrofa (wild boar Mesihan) | http://ftp.ensembl.org/pub/release- |
| | 103/fasta/sus_scrofa_meishan/cdna/ |
| | Sus_scrofa_meishan.Meishan_pig_v1.cdna.all.fa.gz |
| 80. Sus scrofa (wild boar Pietrain) | http://ftp.ensembl.org/pub/release: |
| | 103/fasta/sus_scrofa_pietrain/cdna/ |
| | Sus_scrofa_pietrain.Pietrain_pig_v1.cdna.all.fa.gz |
| 81. Sus scrofa (wild boar | http://ftp.ensembl.org/pub/release- |
| Rongchang) | 103/fasta/sus_scrofa_rongchang/cdna/ |
| | Sus_scrofa_rongchang.Rongchang_pig_v1.cdna.all.fa.gz |
| 82. Sus scrofa (wild boar Tibetan) | http://ftp.ensembl.org/pub/release- |
| | 103/fasta/sus_scrofa_tibetan/cdna/ |
| | Sus_scrofa_tibetan.Tibetan_Pig_v2.cdna.all.fa.gz |
| 83. Sus scrofa (wild boar | http://ftp.ensembl.org/pub/release- |
| Wuzhishan) | 103/fasta/sus_scrofa_wuzhishan/cdna/ |
| | Sus_scrofa_wuzhishan.minipig_v1.0.cdna.all.fa.gz |
| 84. sus scrofa (wild boar | http://ftp.ensembl.org/pub/release- |
| USMARC) | 103/fasta/sus_scrofa_usmarc/cdna/ |
| | Sus_scrofa_usmarc.USMARCv1.0.cdna.all.fa.gz |
| 85. Oryctolagus cuniculus (rabbit) | http://ftp.ensembl.org/pub/release- |
| | 103/fasta/oryctolagus_cuniculus/cdna/ |
| | Oryctolagus_cuniculus.OryCun2.0.cdna.all.fa.gz |
| 86. Rattus norvegicus (rat) | http://ftp.ensembl.org/pub/release- |
| | 103/fasta/rattus_norvegicus/cdna/ |
| | Rattus_norvegicus.Rnor_6.0.cdna.all.fa.gz |
| 87. Saccharomyces cerevisiae | http://ftp.ensembl.org/pub/release- |
| (yeast) | 103/fasta/saccharomyces_cerevisiae/cdna/ |
| | Saccharomyces_cerevisiae.R64-1-1.cdna.all.fa.gz |
| 88. Ovis aries (sheep) | http://ftp.ensembl.org/pub/release- |
| | 103/fasta/ovis_aries_rambouillet/cdna/ |
| | Ovis_aries_rambouillet.Oar_rambouillet_v1.0.cdna.all.fa.gz |
| 89. Meleagris gallopavo (turkey) | http://ftp.ensembl.org/pub/release- |
| | 103/fasta/meleagris_gallopavo/cdna/ |
| | Meleagris_gallopavo.Turkey_2.01.cdna.all.fa.gz |

The present invention further relates to the modified RNA molecule as claimed for use in therapy, diagnosis or prophylaxis.
In vitro and in vivo studies performed by the inventors have shown that immunogenicity of mRNA having a reduced cytidine and optionally reduced uridine content is decreased and its translation is enhanced. This has been demonstrated for e.g. the proteins GFP, secreted nanoluciferase, murine EPO, human IL-15, bone morphogenetic protein 2 (BMP-2), and IL-15 receptor in which cytidines and uridines are replaced by other non-modified nucleotides.
The RNA molecules of the invention are therefore useful in many therapies.
   Therapy can for example be based on the replacement of absent and defective biologically active polypeptides or proteins, supplementation of an endogenous protein to enhance cellular processes counteracting a disorder or repress cellular processes causing a disorder, introduction of non-endogenous biologically active proteins in a patient. Examples of disorders that potentially benefit from treatment with a modified RNA molecule according to the invention include chronic kidney disease, focal segmental glomerulo sclerosis, lupus nephritis, glomerulonephritis, membranoproliferative glomerulonephritis, interstitial nephritis, IgA nephropathy (Berger's disease), pyelonephritis, Goodpasture's syndrome, Wegener's granulomatosis, acute kidney disease, kidney transplant rejection, inflammatory bowel disease, ulcerative colitis, Crohn's disease, coeliac disease, atopic dermatitis, psoriasis, eczema, Behçet's disease, acne, pyoderma, rosacea, systemic lupus erythematosus, asthma, chronic obstructive pulmonary disease, COPD, pneumonitis, rheumatoid arthritis, periodontitis, sinusitis, transplant rejection, ischemia reperfusion injury (also known as reperfusion injury), atherosclerosis, vasculitis, dry eye disease, Sjögren syndrome, corneal vascularization, inflammatory cornea disorders, diabetic nephropathy, sepsis, liver fibrosis/cirrhosis.

Diagnostic purposes for which the modified RNA molecule of the invention can be used include for example detecting specific cells, detecting the presence of proteins, in particular immune suppressor proteins, proteins signaling inflammation, fibrosis and/or cell-stress.

According to the invention, mRNA can be used on cells isolated from a patient to determine the (residual) presence of specific biomolecules or pathways. For example, a reporter mRNA may be used to establish the level of a specific miRNA in the cell. In such an assay, the level of protein expression would be the outcome measure of interest. Therefore, protein expression may not be hindered (in an unpredictable manner and/or magnitude) by innate immunity, a problem avoided by the use of the method of invention.

The modified RNA molecule can be used in prevention, for example as a vaccine, in particular a vaccine against viruses, such as influenza viruses or corona viruses.

The present invention can for example be used for the detection of the absence of tumor suppression. For this a C- and optionally U-depleted mRNA is designed that encodes a fluorescent protein and the 5′UTR and/or 3′UTR region of which comprises a target sequence for the p53 protein. If p53 is present and binds to the region it prevents the gene encoding the fluorescent protein to be translated. No fluorescence is visible if tumor suppression is intact. However, in cases where tumor suppression is impaired the fluorescent protein can be translated and becomes visible as fluorescence in the cell.

The C- and optionally U-depleted mRNA molecules of the invention can be used in treating disorders that involve an inflammatory component. For this, the mRNA encodes an anti-inflammatory protein. Such mRNA can be administered systemically or locally by injection. It may be administered together with a targeting ligand to deliver the mRNA to the location in need of treatment.

In another application, C- and optionally U-depleted mRNA molecules of the invention can be used to prevent or quell a cytokine storm and inflammation resulting from an uncontrolled antiviral response.

A C- and optionally U-depleted mRNA molecule of the invention encoding erythropoietin (EPO) can be administered to blood donors prior to blood donation, or to patients with a chronically low EPO production as a consequence of chronic kidney disease, to promote the formation of red blood cells.

A C- and optionally U-depleted mRNA molecule of the invention can be used in treating disorders arising from insufficient growth or cell-cycling. For these indications, mRNA encoding a growth factor, bone morphogenic factor or cell cycle promoting factor may be used. Such mRNA can be administered systemically or locally by injection. It may be administered together with a targeting ligand to deliver the mRNA to the location in need of treatment.

The modified RNA molecule of the invention is obtainable by the method disclosed herein. In a further embodiment, the modified RNA molecule is the product of the method of the invention.

The present invention further relates to a pharmaceutical composition comprising the modified RNA molecule as claimed. This pharmaceutical composition can be applied for the same uses as defined above.

The invention further relates to the use of the modified RNA molecule in genome editing, for example for producing guide RNAs or RNA-guided endonucleases in CRISPR applications. In one method, both the guide RNA and the CRISPR-cas9 (or related) endonuclease protein are both (optionally simultaneously) introduced in the form of RNA. The CRISPR-cas9 protein could be encoded by an mRNA produced/modified with the method of invention, to obtain a less toxic treatment, a higher expression of CRISPR-cas9 protein and thus a higher genome modification efficacy.

Similarly, when Zinc-finger or other protein-based genome editors are introduced into the cell, mRNA could be a very efficient and highly controllable delivery method. The use of a de-immunized mRNA provides the benefit of higher protein expression and lower cellular toxicity, thus a higher genome editing efficiency.

The method of the invention can be performed in two ways. In a first embodiment, codons are exchanged in a random fashion. Alternatively, codons are exchanged in the order of their appearance in the coding sequence. Preferably, codons are exchanged with alternative codons that occur with the highest frequency in the human genome.

It is preferred that the available alternative codon comprising less cytidine nucleotides encodes the same amino acid. Alternatively, the available alternative codon comprising less cytidine nucleotides result in conservative replacement of the encoded amino acid.

Preferably, the codons are exchanged according to any one of the codon exchange tables 1A, 1B, 2A, 2B, 2C, 2D. Tables 4, 5A and 5B are used in the GU depletion experiments that were used as comparison to the present C-, U- and CU-depletion experiments.

In one embodiment, the method of the invention starts with the modification of the sequence of a DNA molecule that encodes a polypeptide or protein of interest. For this, the coding sequence of the DNA molecule is determined. The coding sequence runs from the start codon ATG and ends with the first in-frame stop codon that occurs. This sequence is then divided in separate codons which together represent the amino acid sequence of the polypeptide or protein of interest. Subsequently, it is determined which codons need modification to remove cytidine and optionally thymidine residues. DNA contains thymidine nucleotides where RNA contains uridine nucleotides. Uridine depletion at the DNA level thus comprises removal of thymidine residues.

The present invention thus relates to a method for reducing the immunogenicity of an RNA molecule and correspondingly enhance protein translation thereof by changing the sequence. Such modified RNA sequences are preferably contacted with cells, preferably eukaryotic cells, in a manner that results in uptake in a proper compartment in the cell, preferably the cytosol, and subsequent modification of cellular behaviour via either peptide or protein expression, or modifying protein behaviour, or modifying RNA behaviour, or a combination thereof. The modification of cellular behaviour is useful for therapeutic, diagnostic or research purposes.

According to the invention, the coding sequence of the messenger RNA of a wild-type protein sequence is selected and for one or more, preferably all suitable, in-frame codons an alternative codon encoding for the same amino acid, and containing less cytidine nucleosides, is selected and the corresponding nucleotide sequence is exchanged. This is called C-depletion.

In a further embodiment of the invention, the coding sequence of the messenger RNA of a wild-type protein sequence is selected and for one or more, preferably all suitable, in-frame codons an alternative codon encoding for the same amino acid, and containing less uridine and cytidine nucleosides, is selected and the corresponding nucleotide sequence is exchanged. This is called UC-depletion.

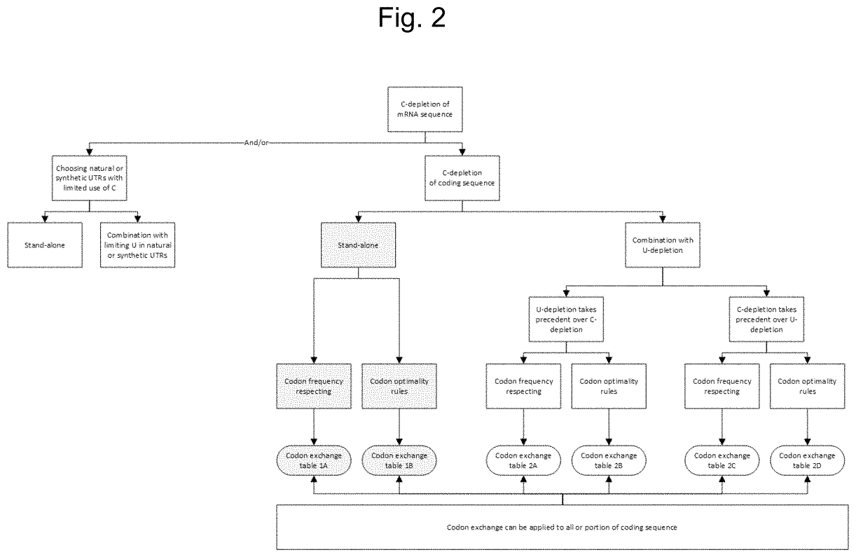

When selecting an alternative codon to replace the original in-frame codon in the nucleotide sequence, preferably the following rules are followed:
1) The alternative codon must be encoding the same amino acid to obtain a wild-type protein.
2a) The alternative codon must have a lower cytidine content, or
2b) The alternative codon must have a lower uridine and/or cytidine content
3) In relation to rule 2b) two options are available:
   a. the uridine content reduction takes precedent over cytidine content reduction if multiple options exist (for example: UUU is replaced with UUC (both amino acid F), or UCU can be replaced with AGC (both amino acid S)).
   b. the cytidine content reduction takes precedent over uridine content reduction if multiple options exist (for example. UUC is replaced with UUU (both amino acid F), or UCU can be replaced with AGU (both amino acid S)).
4) In relation to rule 2a and b, and 3, the exchange of the alternative codon can be conditional on the relative frequency of the codon in the protein coding portion of the genome of the organism of interest, or the relative frequency of the corresponding tRNA in the organism of interest. Here multiple options also exist:
   a. codons are only exchanged if the relative frequency is close to the relative frequency of the original codon. Close being defined as less than 5/1000 codons difference;
   b. codons are only exchanged to alternative codons with the closest relative frequency if multiple options exist (his rule is subordinate to rules 2a and b, and 3);
   c. codons are exchanged according to rules 2, 3 and 4, and selecting the alternative codon with the highest relative frequency. As a variation, codons for which no lower cytidine and/or uridine content alternative codon is available may be also exchanged to an alternative codon with a higher relative frequency.
These rules a schematically illustrated in FIG. 2.
   When applying the rules for the base-use of the RNA, several variants can be thought off. First of all, depletion of cytidine or uridine-cytidine can be applied to the coding sequence by exchanging synonymous codons. For this purpose, there are several codon exchange tables that govern the rules of this exchange. Codon optimality refers to many associations of bias in codon use, tRNA availability, etc. In this manuscript codon optimality is assumed to be related to codons that are more frequently used in coding sequences in the human genome. Codon frequency respecting codon exchange tables assume that the natural codon frequency distribution of the coding sequence is optimal or required for folding of the resulting nascent polypeptide. In these tables codon exchange follows the rules: U-depletion>C-depletion>most similar codon frequency.

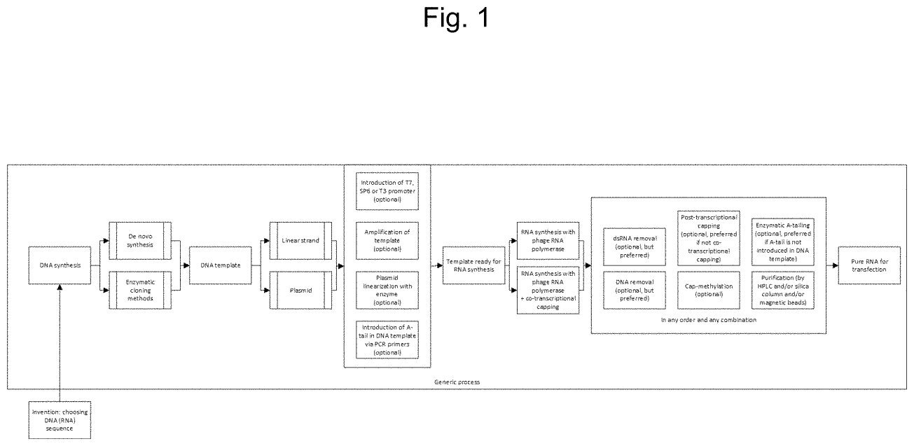

To produce mRNA, an enzymatic process is used (called herein the generic process) that converts the chosen DNA sequence into RNA. Additional post-transcriptional or co-transcriptional enzymatic reactions are used to modify the nascent RNA strand and convert it into messenger RNA. The invention relates to the choosing of the DNA sequence to be produced, which will serve as template for the RNA synthesis. FIG. 1 schematically shows the generic process of how modified RNA for transfection can be prepared after the modification step has taken place. First, the corresponding DNA of an RNA molecule to be modified is chosen. This DNA molecules encodes a polypeptide or protein of interest. The modification can be a virtual modification when the exchange of codons is performed in silico to provide the sequence of a DNA molecule that is then prepared by de novo synthesis. Alternatively, enzymatic cloning methods using restriction enzymes can be used. In addition, combinations of these types of modification can be used. It is for example possible to synthesize one part of the DNA template de novo and combine it with existing 5′UTR and 3′UTR regions.

When the DNA template is prepared it can be used as a linear strand or in a plasmid. Optional steps include addition of a promoter, amplification of the template to increase the amount of template, enzymatically linearizing the plasmid when the DNA template is incorporated in a plasmid and introduction of an A-tail in the DNA template, for example via PCR primers. After these steps the template is ready for RNA synthesis.

Transcription can take place with or without co-transcriptional capping and is followed by one or more optional steps as shown in FIG. 1.

In this application reference is made to the following figures:

FIG. 1 is a schematic representation of the generic process for producing mRNA. It describes the different options and routes how mRNA containing the invention may be prepared.

FIG. 2 is a schematic, detailing the routes by which the invention can be applied to a given sequence.

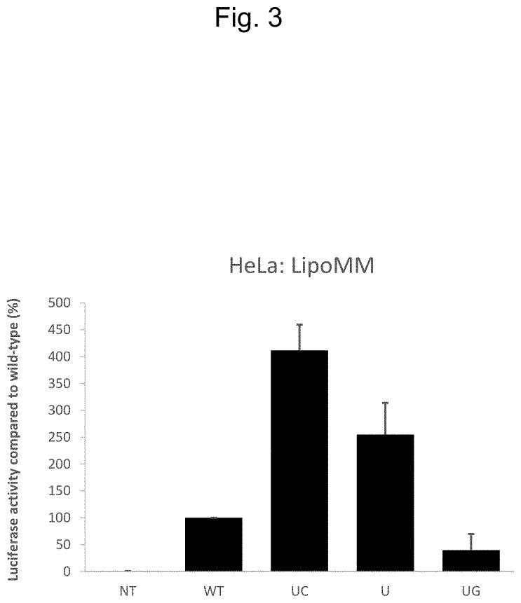

FIG. 3 shows levels of secreted nanoluciferase protein in cell culture medium at 24 h following transfection of 100 ng nanoluciferase coding mRNA, which were prepared according to example 1. The highest protein expression was induced by the UC-depleted mRNA, achieving over 4× the protein expression of the wild-type mRNA. The UC-depleted mRNA translated also significantly more efficient than the U-depleted mRNA, showing the superiority of decreasing the Cytidine content in combination with reducing the Uridine content, compared to reducing the Uridine content only. This experiment demonstrates the additive effect of both modifications. Interestingly, depletion of both Uridine and Guanosine simultaneously did not lead to a higher protein expression, but rather a lower expression compared to WT. This points to the importance of the identity of the Cytidine as the nucleotide that is to be reduced for optimal expression. This is surprising because both Uridine and Guanosine are promiscuous base-pairing partners and have previously been indicated to contribute to TLR7/8 activation, as well as the formation of intramolecular dsRNA formation.

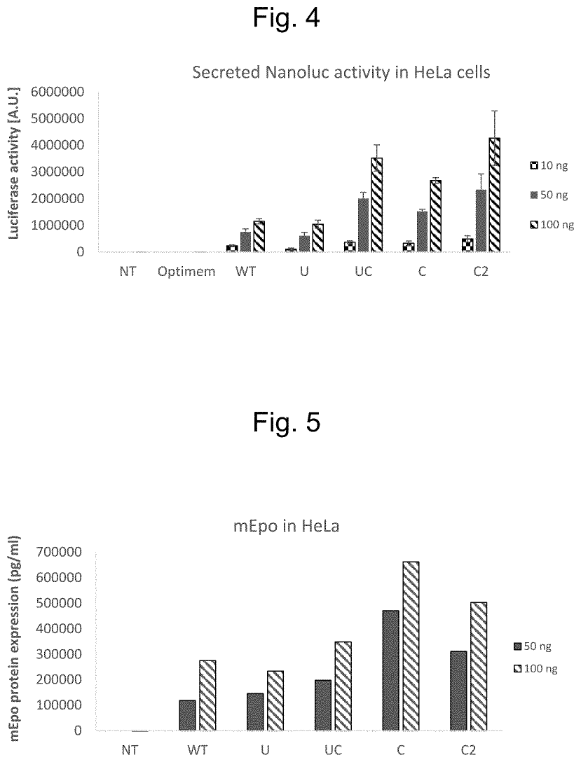

FIG. 4 shows levels of secreted nanoluciferase protein in cell culture medium at 24 h following transfection of 10 ng, 50 ng or 100 ng nanoluciferase coding mRNA, respectively. The mRNAs were prepared according to example 1. The result follow the same trend as those presented in FIG. 3, except that Uridine-depletion does not show significantly improved luciferase expression compared to WT. UC-depleted mRNA translates more efficiently into protein than unmodified WT mRNA. Interestingly, depletion of Cytidine from the nucleic acid sequence shows a much higher protein expression compared to WT, rivalling the levels obtained with UC-depleted mRNA. Furthermore, the results point to a dose-dependent effect of C-depletion, because the C-depleted mRNA, having a lower reduction in Cytidine than C2-depleted mRNA, is expressed at a lower level than C2-depleted mRNA.

FIG. 5 shows levels of secreted murine EPO protein in cell culture medium at 24 h following transfection of 50 or 100 ng of mEPO coding mRNA, respectively. The mRNAs were prepared according to example 1. For mEPO, the benefit from U-depletion and UC-depletion is less pronounced in this experiment and only visible at 50 ng, but C-depletion shows significant higher expression of the protein at all doses. This experiment confirms that the observations, and benefits from C-depletion or UC-depletion hold true for multiple RNA sequences, pointing to a general mechanism.

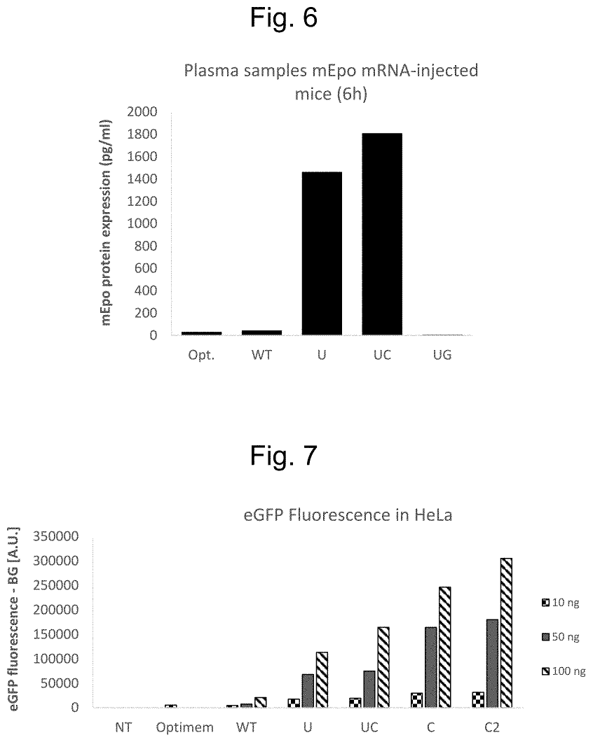

FIG. 6 shows levels of secreted murine EPO protein in mouse plasma collected 6 h after intraperitoneal injection of 1 μg mEPO mRNA complexed with TransIT (Mirus Bio, Madison, Wis.) according to example 3. A high expression of mEPO was found after 6 h for both U-depleted and for UC-depleted mRNA, but barely any mEPO expression was found for the WT and UG-depleted mRNA. This experiment indicates that the depletion of immunogenic nucleotides and sequences in the mRNA is even more important in vivo than in HeLa cells, as the WT mEPO mRNA produced significant amounts of mEPO protein in vitro. Furthermore, this experiment shows the enormous benefit in terms of efficacy of an mRNA therapeutic that can be obtained from the reduction of immunogenic nucleotides, being Uridine and Cytidine, from the sequence.

FIG. 7 shows background-corrected levels of eGFP protein fluorescence obtained from lysed HeLa cells, 24 h after transfection with eGFP mRNA produced according to example 1. The experiment shows a clear increase in protein expression for U-depleted eGFP mRNA. However, addition of C-depletion to the U-depletion increased expression even more, whereas the highest protein expression levels were obtained with C-depletion without changes to the Uridine content. This experiment confirms the generalized effect of the reduction of Cytidine in the sequence on protein expression.

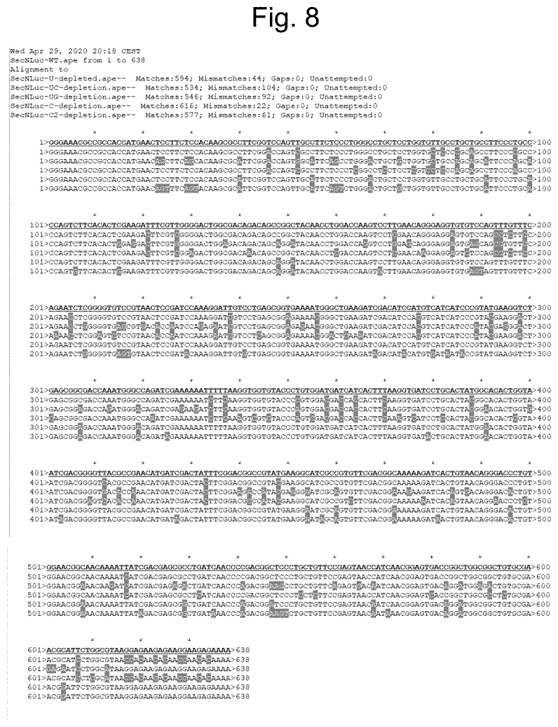

FIG. 8 shows the location and identity of nucleotides changed according to the invention in the RNA sequences coding for secreted nanoluciferase (secNLuc) protein compared to the wild-type mRNA, U-depleted mRNA and UG-depleted mRNA. Changes compared to wild-type (WT) are indicated in a grey box.

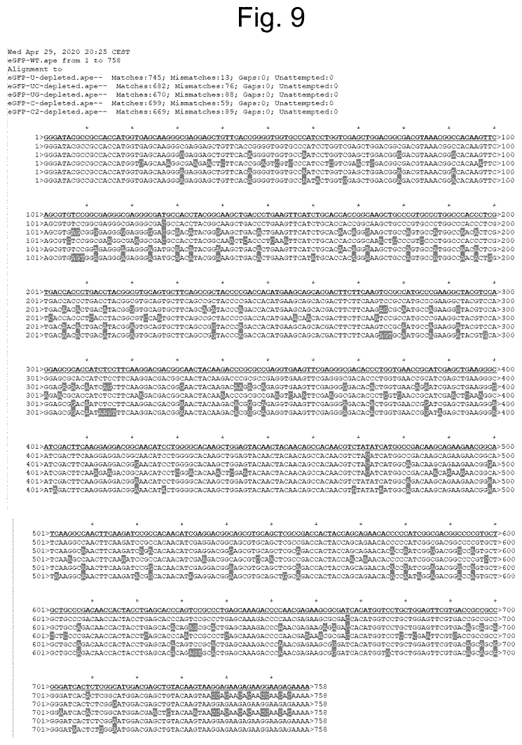

FIG. 9 shows the location and identity of nucleotides changed according to the invention in the RNA sequences coding for enhanced green fluorescent protein (eGFP) protein compared to the wild-type mRNA, U-depleted mRNA and UG-depleted mRNA. Changes compared to wild-type (WT) are indicated in a grey box.

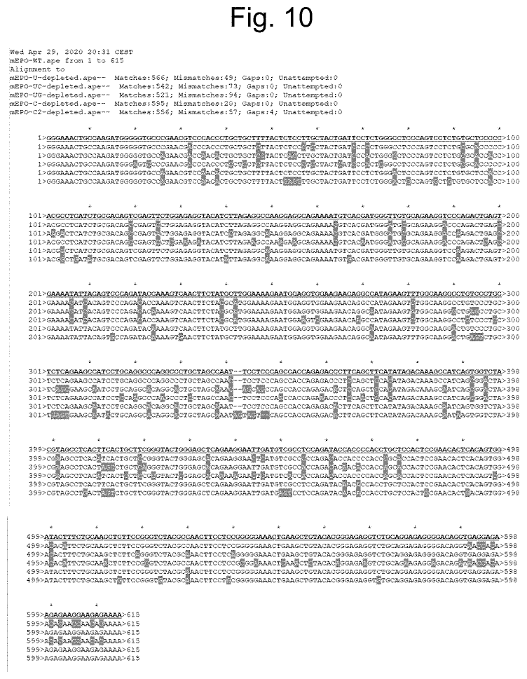

FIG. 10 shows the location and identity of nucleotides changed according to the invention in the RNA sequences coding for murine Erythropoietin (mEPO) protein compared to the wild-type mRNA, U-depleted mRNA and UG-depleted mRNA. Changes compared to wild-type (WT) are indicated in red.

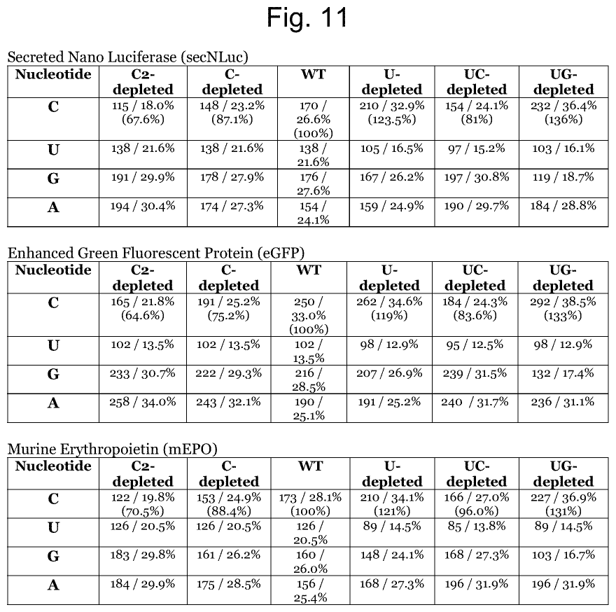

FIG. 11 shows the nucleotide composition of the mRNA sequences in absolute numbers and as percentage. For the number of Cytidines, the percentage compared to the wild-type sequence (set at 100%) is given for comparison.

The present invention will be further illustrated in the Example that follows.

### EXAMPLES

### Example 1

### Sequence-Engineering of mRNAs According to the Invention

To obtain an mRNA according to the invention, first the wildtype DNA sequence of the gene of interest is obtained from sources known to a person skilled in the art. Next, the coding sequence is isolated by identification of the start-codon and in-frame stop-codon according to information from literature, provided by the manufacturer or other methodologies known to a person skilled in the art. For secreted nanoluciferase the coding sequence (Coding sequence 1) was obtained from the manufacturer (Promega). For murine Erythropoietin (mEPO), the coding sequence (Coding sequence 2) was obtained from NCBI (NCBI Reference Sequence: NM_007942.2). For enhanced green fluorescent protein (eGFP), the sequence was previously developed in-house based on literature (Coding sequence 3).

Next, the coding sequence was modified according to the invention. For this, the coding sequence was divided in codons according to methods known to the person skilled in the art. Next, each codon identified in the WT-sequence present in the column named ‘Original codon’ was exchanged with the corresponding codon from the column named ‘Swap codon’ from the corresponding codon exchange table. For codons not present in the column name ‘Original codon’ no changes were made. In this study, for the U-depleted variants of secNLuc, mEPO and eGFP, codon exchange table 4A was used. In this study, for the UC-depleted variants of secNLuc, mEPO and eGFP, codon exchange table 2C was used. In this study, for the UG-depleted variants of secNLuc, mEPO and eGFP, codon exchange table 3 was used. In this study, for the C2-depleted variants of secNLuc, mEPO and eGFP, codon exchange table 1A was used. In this study, for the C-depleted variants of secNLuc, mEPO and eGFP, codon exchange table 5 was used. Next, the desired 5′UTR and 3′UTR (detailed in Table 1 in Example 2) were added in silico to obtain the (modified) RNA sequences obtained from the previous step. The 5′UTR was added directly upstream of the (modified) coding sequence, and the 3′UTR was added directly downstream of the (modified) coding sequence. Next, a T7 promoter (sequence TAATACGACTCACTATA (SEQ ID No.1) followed by up to 3 G nucleotides were added in silico to the 5′ end of the sequence. If the selected 5′UTR already had one or more consecutive Guanosine nucleotides at the 5′end, the number of additional Guanosine nucleotides was reduced so that 3 Guanosine nucleotides remain at the 5′end of the 5′UTR and directly downstream of the T7 promoter sequence. Upstream of the obtained sequence, additional nucleotides were added to facilitate accurate de novo DNA synthesis. For this study, 2 nucleotides (GG) were added in silico upstream of the T7 promoter. Downstream of the obtained sequence, additional nucleotides were added to facilitate de novo DNA synthesis. Additional downstream nucleotides were removed in Example 2 by using reverse PCR primers that start exactly at the desired 3′end.

### Example 2

Generation and Purification of mRNAs

For the wild-type sequence, secreted nano-luciferase DNA was ordered from Promega as a plasmid (pNL3.3). To obtain a linear template, the plasmid was amplified with primers (Fwd primer: tacgtagcgcTAATACGACTCAC (SEQ ID No.2) & Rvs primer: GTATCTTATCATGTCTGCTCGAAG (SEQ ID No.3)) by the Q5 DNA polymerase (annealing temperature 63° C., extension temperature 72° C., annealing time 30 seconds, extension time 20 seconds, 25 cycles of amplification, 2 minute final extension, 10 ng DNA input). Subsequently, the plasmid DNA was digested with DpnI (provide by New England Biolabs (NEB)) for 1 h at 37° C. by adding 20 U of DpnI (1 μl) to the PCR reaction. The digested plasmid DNA and PCR reaction salts and proteins were removed by a Qiagen MinElute PCR cleanup column (Qiagen) according to manufacturer's protocol. The purified DNA template was spectrophotometrically quantified, diluted to 100 ng/μl. mRNA produced from this template was used to validate the luciferase assay.

For the sequence-engineered mRNAs encoding secreted nanoluciferase and the corresponding wild-type control was DNA encoding the secreted nanoluciferase ordered from and synthesized by IDT (Integrated DNA technologies). The delivered DNA was amplified by PCR with primers (Fwd primer: ggaggTAATACGACTCACTATAGGG (SEQ ID No.4) & Rvs primer: TTTTGTGTTGGTTGTGTTGTGGT (SEQ ID No.5) for the U-depleted and UG-depleted mRNA, or Fwd primer: ggaggTAATACGACTCACTATAGGG (SEQ ID No.4) & Rvs primer: TTTTCTCTTCCTTCTCTTCTCCT (SEQ ID No.6) for the WT, UC-depleted and C-depleted mRNAs) and the Q5 DNA polymerase, according to manufacturer's protocol (annealing temperature 63° C., extension temperature 72° C., annealing time 30 seconds, extension time 20 seconds, 25 cycles of amplification, 2 minute final extension, 10 ng DNA input). PCR reaction salts and proteins were removed by a Qiagen MinElute PCR cleanup column (Qiagen) according to manufacturer's protocol. The purified DNA template was spectrophotometrically quantified, diluted to 100ng/μl.

200ng of each of the DNA templates was used as input in a standard T7 RNA polymerase in vitro transcription reaction (according to protocol, NEB HiScribe T7 RNA synthesis kit), including 1 μl of Murine RNAse inhibitor (NEB) per 20 μl of reaction volume to prevent RNAse-mediated degradation of the nascent RNA. The 4 canonical nucleotides (ATP, CTP, UTP, GTP) were used for transcription.

After 3 h incubation at 37° C., 1 μl of Turbo DNAse (2units, Thermo Fisher Scientific) was added and incubated for 1 h at 37° C. Next, the RNA was A-tailed by E.coli poly(A) polymerase (NEB, according to protocol) to obtain a 150 nt-long polyA-tail. After verification of proper A-tail length, the RNA was purified on RNeasy mini silica columns according to manufacturer's protocol (Qiagen). The purified RNA was twice eluted in 2 times 7 μl of RNase-free MQ and spectrophotometrically quantified. Next, a 5′cap (cap1) was added with vaccinia capping enzyme (NEB) and simultaneous 2′O-methyltransferase (NEB) treatment according to manufacturer's protocol. The completed mRNA was purified on cellulose column (according to Baiersdörfer, M. et al. A Facile Method for the Removal of dsRNA Contaminant from In Vitro-Transcribed mRNA. Mol. Ther. —Nucleic Acids 15, 26-35 (2019)) to remove dsRNA arising as side-product from the T7 reaction. The eluate was subsequently purified on a Qiagen RNeasy mini column (first step is to add 1470 μl RLT buffer (Qiagen) and 970 μl 100% ETOH (Sigma Aldrich, >99.8%) and add entire mixed volume in steps of 700 μl to the column and elute). Subsequent steps were according to manufacturer's protocol) and the mRNA was eluted in RNAse-free water. The material was spectrophotometrically quantified and diluted to 1 μg/μl with RNase-free MQ.

All other mRNAs used in this application were synthesized with the method described above, using the 5′UTR and 3′UTR, primers and PCR conditions as shown in Table 1 below.

TABLE 1
Synthesis details of DNA templates for RNA transcription

| | | | | | PCR |
|---|---|---|---|---|---|
| mRNA | 5′UTR | 3′UTR | Fwd primer | Rvs primer | conditions |
| Secreted | GGGAAACGCCGCCACC | GGAGAAGAGAAGGAAGAGAAAA | ggTAATACGACTCACTATAGGG | TTTTCTCTTCCTTCTCTTCTCCT | Anneal: 63° C. |
| nanoluc- | (SEQ ID No. 7) | (SEQ ID No. 8) | (SEQ ID No. 4) | (SEQ ID No. 12) | |
| WT | | | | | |
| Secreted | GGGAAACGCCGCCACC | CCACAACACAACCAACACAAAA | ggTAATACGACTCACTATAGGG | TTTTGTGTTGGTTGTGTTGTGGT | Anneal: 63° C. |
| nanoluc- | (SEQ ID No. 7) | (SEQ ID No. 9) | (SEQ ID No. 4) | (SEQ ID No. 13) | |
| U- | | | | | |
| depleted | | | | | |
| Secreted | GGGAAACGCCGCCACC | GGAGAAGAGAAGGAAGAGAAAA | ggTAATACGACTCACTATAGGG | TTTTCTCTTCCTTCTCTTCTCCT | Anneal: 63° C. |
| nanoluc- | (SEQ ID No. 7) | (SEQ ID No. 8) | (SEQ ID No. 4) | (SEQ ID No. 12) | |
| UC- | | | | | |
| depleted | | | | | |
| Secreted | GGGAAACGCCGCCACC | GGAGAAGAGAAGGAAGAGAAAA | ggTAATACGACTCACTATAGGG | TTTTCTCTTCCTTCTCTTCTCCT | Anneal: 63° C. |
| nanoluc- | (SEQ ID No. 7) | (SEQ ID No. 8) | (SEQ ID No. 4) | (SEQ ID No. 12) | |
| C- | | | | | |
| depleted | | | | | |
| Secreted | GGGAAACGCCGCCACC | GGAGAAGAGAAGGAAGAGAAAA | ggTAATACGACTCACTATAGGG | TTTTCTCTTCCTTCTCTTCTCCT | Anneal: 63° C. |
| nanoluc- | (SEQ ID No. 7) | (SEQ ID No. 8) | (SEQ ID No. 4) | (SEQ ID No. 12) | |
| C2- | | | | | |
| depleted | | | | | |
| Secreted | GGGAAACGCCGCCACC | CCACAACACAACCAACACAAAA | gg TAATACGACTCACTATAGGG | TTTTGTGTTGGTTGTGTTGTGGT | Anneal: 63° C. |
| nanoluc- | (SEQ ID No. 7) | (SEQ ID No. 9) | (SEQ ID No. 4) | (SEQ ID No. 13) | |
| UG- | | | | | |
| depleted | | | | | |
| Murine | GGGAAACTGCCAAG | GGAGAAGAGAAGGAAGAGAAAA | ggTAATACGACTCACTATAGGG | TTTTCTCTTCCTTCTCTTCTCCT | Anneal: 63° C. |
| EPO- | (SEQ ID No. 10) | (SEQ ID No. 8) | (SEQ ID No. 4) | (SEQ ID No. 12) | |
| WT | | | | | |
| Murine | GGGAAACTGCCAAG | CCACAACACAACCAACACAAAA | ggTAATACGACTCACTATAGGG | TTTTGTGTTGGTTGTGTTGTGGT | Anneal: 63° C. |
| EPO- | (SEQ ID No. 10) | (SEQ ID No. 9) | (SEQ ID No. 4) | (SEQ ID No. 13) | |
| U- | | | | | |
| depleted | | | | | |
| Murine | GGGAAACTGCCAAG | GGAGAAGAGAAGGAAGAGAAAA | ggTAATACGACTCACTATAGGG | TTTTCTCTTCCTTCTCTTCTCCT | Anneal: 63° C. |
| EPO- | (SEQ ID No. 10) | (SEQ ID No. 8) | (SEQ ID No. 4) | (SEQ ID No. 12) | |
| UC- | | | | | |
| depleted | | | | | |
| Murine | GGGAAACTGCCAAG | GGAGAAGAGAAGGAAGAGAAAA | ggTAATACGACTCACTATAGGG | TTTTCTCTTCCTTCTCTTCTCCT | Anneal: 63° C. |
| EPO- | (SEQ ID No. 10) | (SEQ ID No. 8) | (SEQ ID No. 4) | (SEQ ID No. 12) | |
| C- | | | | | |
| depleted | | | | | |
| Murine | GGGAAACTGCCAAG | GGAGAAGAGAAGGAAGAGAAAA | ggTAATACGACTCACTATAGGG | TTTTCTCTTCCTTCTCTTCTCCT | Anneal: 63° C. |
| EPO- | (SEQ ID No. 10) | (SEQ ID No. 8) | (SEQ ID No. 4) | (SEQ ID No. 12) | |
| C2- | | | | | |
| depleted | | | | | |
| EGFP- | GGGATACGCCGCCACC | GGAGAAGAGAAGGAAGAGAAAA | ggTAATACGACTCACTATAGGG | TTTTCTCTTCCTTCTCTTCTCCT | Anneal: 63° C. |
| WT | (SEQ ID No. 11) | (SEQ ID No. 8) | (SEQ ID No. 4) | (SEQ ID No. 12) | |
| EGFP- | GGGATACGCCGCCACC | CCACAACACAACCAACACAAAA | ggTAATACGACTCACTATAGGG | TTTTGTGTTGGTTGTGTTGTGGT | Anneal: 63° C. |
| U- | (SEQ ID No. 11) | (SEQ ID No. 9) | (SEQ ID No. 4) | (SEQ ID No. 13) | |
| depleted | | | | | |
| EGFP- | GGGATACGCCGCCACC | GGAGAAGAGAAGGAAGAGAAAA | ggTAATACGACTCACTATAGGG | TTTTCTCTTCCTTCTCTTCTCCT | Anneal: 63° C. |
| UC- | (SEQ ID No. 11) | (SEQ ID No. 8) | (SEQ ID No. 4) | (SEQ ID No. 12) | |
| depleted | | | | | |
| EGFP- | GGGATACGCCGCCACC | GGAGAAGAGAAGGAAGAGAAAA | ggTAATACGACTCACTATAGGG | TTTTCTCTTCCTTCTCTTCTCCT | Anneal: 63° C. |
| C- | (SEQ ID No. 11) | (SEQ ID No. 8) | (SEQ ID No. 4) | (SEQ ID No. 12) | |
| depleted | | | | | |
| EGFP | GGGATACGCCGCCACC | GGAGAAGAGAAGGAAGAGAAAA | ggTAATACGACTCACTATAGGG | TTTTCTCTTCCTTCTCTTCTCCT | Anneal: 63° C. |
| C2- | (SEQ ID No. 11) | (SEQ ID No. 8) | (SEQ ID No. 4) | (SEQ ID No. 12) | |
| depleted | | | | | |

N.B. EPO = Erythropoietin, NanoLuc = nanoluciferase, eGFP = enhanced Green Fluorescent. Protein

### Example 3

Preparation of Lipofectamine MessengerMax-Complexed mRNA

Because uptake of naked mRNA into the cytosol is minimal to non-existing in the majority of cells, mRNA was complexed with a delivery vehicle (Lipofectamine Messenger-Max) to facilitate uptake in cells in vitro.

mRNA produced according to Example 2 was complexed with Lipofectamine MessengerMax (ThermoFisherScientific) according to instructions by the manufacturer. Briefly, first mRNA was diluted with sterile Optimem (4° C.) to 20ng/μl and 10 μl of the diluted mRNA was used for a single complexation. 0.3 μl of Lipofectamine MessengerMax reagent was mixed with 10 μl of sterile, pre-warmed (to RT) Optimem medium by pipetting. After 10 minutes of incubation, the entire 10 μl was mixed with 10 μl of pre-diluted mRNA (containing a total of 200 ng of mRNA). After careful mixed by pipetting up and down, the mixed components were incubated for 5 minutes at RT before injection or addition to cell culture.

For complexing different amounts of mRNA, the volumes of reagents and the final volume scaled proportionally.

### Example 4

Preparation of TransIT-Complexed mRNA

Some of the mRNA were complexed with another delivery vehicle (TransIT) to facilitate uptake in cells in vivo.

mRNA produced according to Example 2 was complexed with TransIT (Mirus Bio, Madison, Wis.) according to manufacturer's instructions. Briefly, 1 μg of mRNA (generally 1 μl) was mixed with 98 μl of pre-warmed (to RT) DMEM (Dulbecco's modified Eagle medium), followed by the addition of 1.1 μl of TransIT-mRNA reagent and 0.7 μl of Boost reagent. After combination, the mixture was briefly, gently vortexed and incubated for 2-5 minutes before injection.

For complexing different amounts of mRNA, the volumes of reagents and the final volume scaled proportionally.

### Example 5

Administration of Formulated mRNAs

The day before transfection, HeLa cells were plated in 96-well plate at 40% confluency (100 μl of medium (DMEM +10% FCS)/well). 24 h later, HeLa cells, grown to 80% confluency in a 96-well plate were transfected with 10, 50 or 100 ng of secNLuc mRNA complexed with Lipofectamine MessengerMax (Thermo Fisher Scientific) prepared according to Example 3 and incubated for 24 h at 37° C. In case of 100 ng, 10 μl of complexed mRNA solution is mixed with 100 μl of medium and added to the cells. In case of 50 ng, 5 μl of complexed mRNA solution is mixed with 5 μl of Optimem medium and then subsequently mixed with 100 μl of medium and added to cells. In case of 10 ng, 1 μl of complexed mRNA solution is mixed with 9 μl of Optimem medium and then subsequently mixed with 100 μl of medium and added to cells.

After incubation, the entire medium volume was removed and a sample was taken for analysis Animal studies were performed in accordance with the Dutch animal welfare regulations and approved by the Central Animal Experiments Committee (VD103002015270). 1 μg of wild-type (WT), U-, UC- or UG-depleted mEpo mRNA was formulated with TransIT (MirusBio) according to Example 4. After mixing, the formulation was incubated for 5 minutes at RT and directly injected into mice. For this, 10-12 week-old female BALB/cJRj mice (Janvier Labs) were intraperitoneally injected with 100 μl of respective mRNA formulation. After 6 and 24 hours, blood was collected via the tail vein in a Heparin-coated capillary tube. Heparin-plasma was transferred to a 1.5-ml Eppendorf tube and stored at −20° C. until further use. Plasma samples were tested for mEpo using the mEpo assay (R&D) as described above using 5-fold dilution of the plasma samples in Calibrator Diluent.

### Example 6

### Detection of Protein Expression

For measuring secreted nanoluciferase, medium was collected 24 hours after transfection. Luciferase activity was detected with the Nano-Glo Luciferase Assay System (Promega) according to manufacturer's specifications. Importantly, the assay buffer was thawed and equilibrated to RT for more than 1 hour at RT.

For measuring eGFP, medium was removed 24 h after transfection and cells were washed twice with PBS and cell lysates were prepared by adding 30 μl lysis buffer (10 mM TrisHCl pH7+10% glycerol, 2% Tween, 2% Triton X-100 and 0.31 mg/ml freshly added DTT) per well. Cell were incubated for 20 minutes at 37° C. and cell lysates were collected and pooled from 3 wells. Fluorescence was measured using a 485/20 excitation and 528/20 emission filter on a plate reader.

mEpo concentrations were measured in supernatant collected 24 hours after transfection, using the mEpo assay (R&D Systems) according to the manufacturer's protocol. In short, 50 μl Assay Diluent was added to pre-coated wells and supplemented with 50 μl prepared standard or supernatants diluted in Calibrator Diluent. Wells were incubated for 2 hours at RT with shaking. Wells were washed 5 times with 200 μl wash buffer and 100 μl Mouse Epo conjugate was added to each well. After incubating for 2 hours at RT with shaking, wells were washed 5 times with 200 μl wash buffer. Wells were developed with 100 μl Substrate Solution per well for 20-30 minutes at RT in the dark, depending on the strength of the signal. The reaction was stopped by adding 100 μl Stop Solution to each well and the signal was measured at 450 nm in a plate reader (Biorad).

MCP-1 was measured in supernatants that were collected after 24 hours, using the mouse MCP-1 ELISA (R&D Systems) according to the manufacturer's protocols. Shortly, a Costar Maxisorb 96-well plate was coated overnight at 4° C. with 100 μl/well Capture Antibody. Wells washed 3× with 250-300 μl/well Wash Buffer (0.05% Tween-20 in PBS) and blocked for 1 hour at RT with 250 μl 1% (98%-pure) BSA in PBS. Subsequently, wells were washed 3× with 250-300 μl Wash Buffer. Pre-diluted samples and recombinant MCP-1 standard was transferred to the wells and incubated for 2 hours at RT. Wells were again washed 5× with 250-300 μl Wash Buffer and incubated for 1 hour at RT with 100 μl/well Detection Antibody. After washing the wells washed 5× with 250-300 μl Wash Buffer, wells were incubated for 30 minutes at RT with 100 μl/well Avidin-HRP, and washed as described above. 100 μl/well TMB Solution was added and incubated for 10-15 minutes. The reaction was stopped by adding 50 μl/well 2 M H2SO4 and measured at 450 nm using a plate reader (Biorad).

### RESULTS OF DEPLETION EXPERIMENTS

As can be seen in FIG. 3, depletion of Uridine by codon exchange increased the expression of nanoluciferase about 2-fold. Since a conservative algorithm was used (matching codons that are exchanged on frequency of occurring in the human coding genome), this effect is not to be expected to be the result from codon optimization, but rather from a reduction of innate immune reactions that, among other effects, reduce protein expression.

Interestingly, the combination of Uridine depletion with Cytidine depletion resulted in even higher protein expression, suggesting an additive effect of Cytidine nucleotides on activation of innate immune receptors.

Surprisingly, combination of Uridine depletion with Guanosine depletion, typically creating an mRNA rich in Cytidine, resulted in a decreased protein expression compared to wild-type. This result is surprising because Uridine and Guanosine are able to bind each other in addition to their preferred binding partners Adenosine and Cytidine, respectively. Reduction of both Uridine and Guanosine would have been expected to reduce innate immunity and thus boost protein expression by reducing the options for extended dsRNA formation in an RNA structure. In addition, several studies (e.g. Zhang, Z. et al. Structural Analysis Reveals that Toll-like Receptor 7 Is a Dual Receptor for Guanosine and Single-Stranded RNA Immunity 45, 737-748 (2016) and Tanji, H. et al. Toll-like receptor 8 senses degradation products of single-stranded RNA. Nat. Struct. Mol. Biol. (2015). doi:10.1038/nsmb.2943) have indicated the Uridine in the presence of Guanosine would be particularly activating for TLR7 and TLR8, two of the innate immune sensors.

In a further experiment, clarification on the role of Cytidine in the reduction of mRNA mediated protein expression was obtained Similar to the previous experiment, UC-depleted mRNA shows a higher expression than U-depleted mRNA. Interestingly, C-depletion by itself also resulted in increased secreted nanoluciferase expression compared to U-depleted and WT mRNA. Further strengthening the case for Cytidine involvement is the dose-response effect that was obtained by further reducing the number of Cytidine nucleotides in C2-depleted mRNA compared to C-depleted mRNA, resulting in even higher protein expression. This effect was maintained across all doses tested.

Similar results were obtained with murine EPO coding mRNA, both Uridine and Cytidine depletion, alone or in combination, resulted in enhanced protein expression in HeLa cells. Intra-peritoneal injection of the mRNAs in mice resulted in significantly increased circulating mEPO plasma levels at 6h after injection for the U-depleted and UC-depleted mRNAs. The differences with wild-type and UG-depleted mRNAs were even greater, suggesting the role of innate immune activation reduction in protein expression from mRNA is greater in vivo than in HeLa cells. Furthermore, it strengthens the case for Cytidine-depletion or UC-depletion mediated de-immunization of mRNAs to be used for therapeutic purposes.

Finally, using eGFP, a similar protein expression effect was observed for depleted mRNAs coding for an intracellular protein. In order of increasing protein expression: WT, U-depleted, UC-depleted, C-depleted and C2-depleted. Interestingly, again the highest protein expression was obtained with C-depleted and C2-depleted mRNAs. The observed effects were maintained over all doses.

### CODING SEQUENCES

Coding sequence 1-WT coding sequence secreted NanoLuc
(SEQ ID No: 14)
ATGAACTCCTTCTCCACAAGCGCCTTCGGTCCAGTTGCCTTCTCCCTGGGCCTGCTCCT
GGTGTTGCCTGCTGCCTTCCCTGCCCCAGTCTTCACACTCGAAGATTTCGTTGGGGACT
GGCGACAGACAGCCGGCTACAACCTGGACCAAGTCCTTGAACAGGGAGGTGTGTCCA
GTTTGTTTCAGAATCTCGGGGTGTCCGTAACTCCGATCCAAAGGATTGTCCTGAGCGG
TGAAAATGGGCTGAAGATCGACATCCATGTCATCATCCCGTATGAAGGTCTGAGCGGC
GACCAAATGGGCCAGATCGAAAAAATTTTTAAGGTGGTGTACCCTGTGGATGATCATC
ACTTTAAGGTGATCCTGCACTATGGCACACTGGTAATCGACGGGGTTACGCCGAACAT
GATCGACTATTTCGGACGGCCGTATGAAGGCATCGCCGTGTTCGACGGCAAAAAGATC
ACTGTAACAGGGACCCTGTGGAACGGCAACAAAATTATCGACGAGCGCCTGATCAAC
CCCGACGGCTCCCTGCTGTTCCGAGTAACCATCAACGGAGTGACCGGCTGGCGGCTGT
GCGAACGCATTCTGGCGTAA
Coding sequence 2-WT coding sequence murine Erythropoietin
(SEQ ID No: 15)
ATGGGGGTGCCCGAACGTCCCACCCTGCTGCTTTTACTCTCCTTGCTACTGATTCCTCTG
GGCCTCCCAGTCCTCTGTGCTCCCCCACGCCTCATCTGCGACAGTCGAGTTCTGGAGAGG
TACATCTTAGAGGCCAAGGAGGCAGAAAATGTCACGATGGGTTGTGCAGAAGGTCCCAG
ACTGAGTGAAAATATTACAGTCCCAGATACCAAAGTCAACTTCTATGCTTGGAAAAGAAT
GGAGGTGGAAGAACAGGCCATAGAAGTTTGGCAAGGCCTGTCCCTGCTCTCAGAAGCCA
TCCTGCAGGCCCAGGCCCTGCTAGCCAATTCCTCCCAGCCACCAGAGACCCTTCAGCTTC
ATATAGACAAAGCCATCAGTGGTCTACGTAGCCTCACTTCACTGCTTCGGGTACTGGGA
GCTCAGAAGGAATTGATGTCGCCTCCAGATACCACCCCACCTGCTCCACTCCGAACACTC
ACAGTGGATACTTTCTGCAAGCTCTTCCGGGTCTACGCCAACTTCCTCCGGGGGAAACTG
AAGCTGTACACGGGAGAGGTCTGCAGGAGAGGGGACAGGTGA
Coding sequence 3-WT coding sequence eGFP
(SEQ ID No: 16)
ATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCGTGCCCTGGC
CCACCCTCGTGACCACCCTGACCTACGGCGTGCAGTGCTTCAGCCGCTACCCCGACCACA
TGAAGCAGCACGACTTCTTCAAGTCCGCCATGCCCGAAGGCTACGTCCAGGAGCGCACC
ATCTCCTTCAAGGACGACGGCAACTACAAGACCCGCGCCGAGGTGAAGTTCGAGGGCGA
CACCCTGGTGAACCGCATCGAGCTGAAGGGCATCGACTTCAAGGAGGACGGCAACATCC
TGGGGCACAAGCTGGAGTACAACTACAACAGCCACAACGTCTATATCATGGCCGACAAG
CAGAAGAACGGCATCAAGGCCAACTTCAAGATCCGCCACAACATCGAGGACGGCAGCGT
GCAGCTCGCCGACCACTACCAGCAGAACACCCCCATCGGCGACGGCCCCGTGCTGCTGC
CCGACAACCACTACCTGAGCACCCAGTCCGCCCTGAGCAAAGACCCCAACGAGAAGCGC
GATCACATGGTCCTGCTGGAGTTCGTGACCGCCGCCGGGATCACTCTCGGCATGGACGA
GCTGTACAAGTAA

### PROTEIN SEQUENCES

All used/sequence-engineered murine EPO (Mus musculus) nucleic acid sequences encode the same murine EPO protein with the following amino acid sequence(SEQ ID No:17) :

MGVPERPTLLLLLSLLLIPL
GLPVLCAPPRLICDSRVLER
YILEAKEAENVTMGCAEGPR
LSENITVPDTKVNFYAWKRM
EVEEQAIEVWQGLSLLSEAI
LQAQALLANSSQPPETLQLH
IDKAISGLRSLTSLLRVLGA
QKELMSPPDTTPPAPLRTLT
VDTFCKLFRVYANFLRGKLK
LYTGEVCRRGDR*

All used/sequence-engineered eGFP (extensively mutated from Aequorea victoria) nucleic acid sequences encode the same eGFP protein with the following amino acid sequence (SEQ ID No:18):

MVSKGEELFTGVVPILVELD
GDVNGHKFSVSGEGEGDATY
GKLTLKFICTTGKLPVPWPT
LVTTLTYGVQCFSRYPDHMK
QHDFFKSAMPEGYVQERTIS
FKDDGNYKTRAEVKFEGDTL
VNRIELKGIDFKEDGNILGH
KLEYNYNSHNVYIMADKQKN
GIKANFKIRHNIEDGSVQLA
DHYQQNTPIGDGPVLLPDNH
YLSTQSALSKDPNEKRDHMV
LLEFVTAAGITLGMDELYK*

All used/sequence-engineered secreted nanoluciferase (developed by Promega) nucleic acid sequences encode the same nanoluciferase protein with the following amino acid sequence (SEQ ID No:19):

MNSFSTSAFGPVAFSLGLLL
VLPAAFPAPVFTLEDFVGDW
RQTAGYNLDQVLEQGGVSSL
FQNLGVSVTPIQRIVLSGEN
GLKIDIHVIIPYEGLSGDQM
GQIEKIFKVVYPVDDHHFKV
ILHYGTLVIDGVTPNMIDYF
GRPYEGIAVFDGKKITVTGT
LWNGNKIIDERLINPDGSLL
FRVTINGVTGWRLCERILA*

### NUCLEIC ACID SEQUENCES

Secreted NanoLuc-WT (assay control)
(SEQ ID No: 20)
GGGATACGCCGCCACCATGAACTCCTTCTCCACAAGCGCCTTCGGTCCAGTTGCCTTCT
CCCTGGGCCTGCTCCTGGTGTTGCCTGCTGCCTTCCCTGCCCCAGTCTTCACACTCGAA
GATTTCGTTGGGGACTGGCGACAGACAGCCGGCTACAACCTGGACCAAGTCCTTGAAC
AGGGAGGTGTGTCCAGTTTGTTTCAGAATCTCGGGGTGTCCGTAACTCCGATCCAAAG
GATTGTCCTGAGCGGTGAAAATGGGCTGAAGATCGACATCCATGTCATCATCCCGTAT
GAAGGTCTGAGCGGCGACCAAATGGGCCAGATCGAAAAAATTTTTAAGGTGGTGTAC
CCTGTGGATGATCATCACTTTAAGGTGATCCTGCACTATGGCACACTGGTAATCGACG
GGGTTACGCCGAACATGATCGACTATTTCGGACGGCCGTATGAAGGCATCGCCGTGTT
CGACGGCAAAAAGATCACTGTAACAGGGACCCTGTGGAACGGCAACAAAATTATCGA
CGAGCGCCTGATCAACCCCGACGGCTCCCTGCTGTTCCGAGTAACCATCAACGGAGTG
ACCGGCTGGCGGCTGTGCGAACGCATTCTGGCGTAAGGCCGCGACTCTAGAGTCGGG
GCGGCCGGCCGCTTCGAGCAGACATGATAAGATAC
Secreted NanoLuc-WT (control to other mRNAs)
(SEQ ID No: 21)
GGGAAACGCCGCCACCATGAACTCCTTCTCCACAAGCGCCTTCGGTCCAGTTGCCTTC
TCCCTGGGCCTGCTCCTGGTGTTGCCTGCTGCCTTCCCTGCCCCAGTCTTCACACTCGA
AGATTTCGTTGGGGACTGGCGACAGACAGCCGGCTACAACCTGGACCAAGTCCTTGAA
CAGGGAGGTGTGTCCAGTTTGTTTCAGAATCTCGGGGTGTCCGTAACTCCGATCCAAA
GGATTGTCCTGAGCGGTGAAAATGGGCTGAAGATCGACATCCATGTCATCATCCCGTA
TGAAGGTCTGAGCGGCGACCAAATGGGCCAGATCGAAAAAATTTTTAAGGTGGTGTA
CCCTGTGGATGATCATCACTTTAAGGTGATCCTGCACTATGGCACACTGGTAATCGAC
GGGGTTACGCCGAACATGATCGACTATTTCGGACGGCCGTATGAAGGCATCGCCGTGT
TCGACGGCAAAAAGATCACTGTAACAGGGACCCTGTGGAACGGCAACAAAATTATCG
ACGAGCGCCTGATCAACCCCGACGGCTCCCTGCTGTTCCGAGTAACCATCAACGGAGT
GACCGGCTGGCGGCTGTGCGAACGCATTCTGGCGTAAGGAGAAGAGAAGGAAGAGAA
AA
Secreted NanoLuc-U-depleted (maximum exchange)
(SEQ ID No: 22)
GGGAAACGCCGCCACCATGAACTCCTTCTCCACAAGCGCCTTCGGACCAGTCGCCTTC
TCCCTGGGCCTGCTCCTGGTGCTCCCCGCAGCCTTCCCCGCCCCAGTCTTCACACTCGA
AGACTTCGTCGGGGACTGGCGACAGACAGCCGGCTACAACCTGGACCAAGTCCTCGA
ACAGGGAGGAGTGTCCAGCCTCTTCCAGAACCTCGGGGTGTCCGTAACTCCGATCCAA
AGGATCGTCCTGAGCGGAGAAAACGGGCTGAAGATCGACATCCACGTCATCATCCCG
TACGAAGGACTGAGCGGCGACCAAATGGGCCAGATCGAAAAAATCTTCAAGGTGGTG
TACCCCGTGGACGACCACCACTTCAAGGTGATCCTGCACTACGGCACACTGGTAATCG
ACGGGGTCACGCCGAACATGATCGACTACTTCGGACGGCCGTACGAAGGCATCGCCG
TGTTCGACGGCAAAAAGATCACTGTAACAGGGACCCTGTGGAACGGCAACAAAATCA
TCGACGAGCGCCTGATCAACCCCGACGGCTCCCTGCTGTTCCGAGTAACCATCAACGG
AGTGACCGGCTGGCGGCTGTGCGAACGCATCCTGGCGTAACCACAACACAACCAACA
CAAAA
Secreted NanoLuc-UC-depleted (maximum exchange)
(SEQ ID No: 23)
GGGAAACGCCGCCACCATGAACAGCTTCAGCACAAGCGCATTCGGACCAGTGGCATT
CAGCCTGGGACTGCTGCTGGTGCTGCCAGCAGCATTCCCAGCACCAGTCTTCACACTG
GAGGACTTCGTGGGGGACTGGAGACAGACAGCAGGATACAACCTGGACCAGGTCCTG
GAGCAGGGAGGAGTGAGCAGCCTGTTCCAGAACCTGGGGGTGAGCGTGACACCAATC
CAGAGAATCGTCCTGAGCGGAGAGAACGGGCTGAAGATCGACATCCACGTCATCATC
CCATACGAGGGACTGAGCGGAGACCAGATGGGACAGATCGAGAAGATCTTCAAGGTG
GTGTACCCAGTGGACGACCACCACTTCAAGGTGATCCTGCACTACGGAACACTGGTGA
TCGACGGGGTGACACCAAACATGATCGACTACTTCGGAAGACCATACGAGGGAATCG
CAGTGTTCGACGGAAAGAAGATCACAGTGACAGGGACACTGTGGAACGGAAACAAGA
TCATCGACGAGAGACTGATCAACCCAGACGGAAGCCTGCTGTTCAGAGTGACAATCA
ACGGAGTGACAGGATGGAGACTGTGCGAGAGAATCCTGGCATAAGGAGAAGAGAAG
GAAGAGAAAA
Secreted NanoLuc-UG-depleted (maximum exchange)
(SEQ ID No: 24)
GGGAAACGCCGCCACCATGAACTCCTTCTCCACAAGCGCCTTCGGACCAGTCGCCTTC
TCCCTCGGCCTCCTCCTCGTCCTCCCAGCAGCCTTCCCAGCCCCAGTCTTCACACTCGA
AGACTTCGTCGGAGACTGGCGACAAACAGCCGGCTACAACCTCGACCAAGTCCTCGA
ACAAGGAGGAGTCTCCAGCCTCTTCCAAAACCTCGGAGTCTCCGTAACACCAATCCAA
AGAATCGTCCTCAGCGGAGAAAACGGACTCAAAATCGACATCCACGTCATCATCCCAT
ACGAAGGACTCAGCGGCGACCAAATGGGCCAAATCGAAAAAATCTTCAAAGTCGTCT
ACCCAGTCGACGACCACCACTTCAAAGTCATCCTCCACTACGGCACACTCGTAATCGA
CGGAGTCACACCAAACATGATCGACTACTTCGGACGCCCATACGAAGGCATCGCCGTC
TTCGACGGCAAAAAAATCACAGTAACAGGAACCCTCTGGAACGGCAACAAAATCATC
GACGAGCGCCTCATCAACCCCGACGGCTCCCTCCTCTTCCGAGTAACCATCAACGGAG
TCACCGGCTGGCGCCTCTGCGAACGCATCCTCGCATAACCACAACACAACCAACACAA
AA
Secreted NanoLuc-C-depleted (maximum exchange of only C-containing
but not U-containing codons)
(SEQ ID No: 25)
GGGAAACGCCGCCACCATGAACTCCTTCTCCACAAGCGCATTCGGTCCAGTTGCATTC
TCCCTGGGACTGCTCCTGGTGTTGCCTGCTGCATTCCCTGCACCAGTCTTCACACTCGA
AGATTTCGTTGGGGACTGGCGACAGACAGCAGGATACAACCTGGACCAAGTCCTTGA
ACAGGGAGGTGTGTCCAGTTTGTTTCAGAATCTCGGGGTGTCCGTAACTCCGATCCAA
AGGATTGTCCTGAGCGGTGAAAATGGGCTGAAGATCGACATCCATGTCATCATCCCGT
ATGAAGGTCTGAGCGGAGACCAAATGGGACAGATCGAAAAAATTTTTAAGGTGGTGT
ACCCTGTGGATGATCATCACTTTAAGGTGATCCTGCACTATGGAACACTGGTAATCGA
CGGGGTTACGCCGAACATGATCGACTATTTCGGACGGCCGTATGAAGGAATCGCAGTG
TTCGACGGAAAAAAGATCACTGTAACAGGGACACTGTGGAACGGAAACAAAATTATC
GACGAGCGGCTGATCAACCCAGACGGATCCCTGCTGTTCCGAGTAACAATCAACGGA
GTGACAGGATGGCGGCTGTGCGAACGGATTCTGGCGTAAGGAGAAGAGAAGGAAGA
GAAAA
Secreted NanoLuc-C2-depleted (maximum exchange of all C-containing codons)
(SEQ ID No: 26)
GGGAAACGCCGCCACCATGAACAGTTTCAGTACAAGCGCATTCGGTCCAGTTGCATTC
AGTCTGGGACTGCTGCTGGTGTTGCCTGCTGCATTCCCTGCACCAGTGTTCACACTGGA
AGATTTCGTTGGGGACTGGCGACAGACAGCAGGATACAACCTGGACCAAGTGCTTGA
ACAGGGAGGTGTGAGTAGTTTGTTTCAGAATCTGGGGGTGAGTGTAACTCCGATACAA
AGGATTGTGCTGAGCGGTGAAAATGGGCTGAAGATAGACATACATGTGATAATACCG
TATGAAGGTCTGAGCGGAGACCAAATGGGACAGATAGAAAAAATTTTTAAGGTGGTG
TACCCTGTGGATGATCATCACTTTAAGGTGATACTGCACTATGGAACACTGGTAATAG
ACGGGGTTACGCCGAACATGATAGACTATTTCGGACGGCCGTATGAAGGAATAGCAG
TGTTCGACGGAAAAAAGATAACTGTAACAGGGACACTGTGGAACGGAAACAAAATTA
TAGACGAGCGGCTGATAAACCCAGACGGAAGTCTGCTGTTCCGAGTAACAATAAACG
GAGTGACAGGATGGCGGCTGTGCGAACGGATTCTGGCGTAAGGAGAAGAGAAGGAA
GAGAAAA
eGFP-WT
(SEQ ID No: 27)
GGGATACGCCGCCACCATGGTGAGCAAGGGCGAGGAGCTGTTCACCGGGGTGGTGCC
CATCCTGGTCGAGCTGGACGGCGACGTAAACGGCCACAAGTTCAGCGTGTCCGGCGA
GGGCGAGGGCGATGCCACCTACGGCAAGCTGACCCTGAAGTTCATCTGCACCACCGG
CAAGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCCCG
AAGGCTACGTCCAGGAGCGCACCATCTCCTTCAAGGACGACGGCAACTACAAGACCC
GCGCCGAGGTGAAGTTCGAGGGCGACACCCTGGTGAACCGCATCGAGCTGAAGGGCA
TCGACTTCAAGGAGGACGGCAACATCCTGGGGCACAAGCTGGAGTACAACTACAACA
GCCACAACGTCTATATCATGGCCGACAAGCAGAAGAACGGCATCAAGGCCAACTTCA
AGATCCGCCACAACATCGAGGACGGCAGCGTGCAGCTCGCCGACCACTACCAGCAGA
ACACCCCCATCGGCGACGGCCCCGTGCTGCTGCCCGACAACCACTACCTGAGCACCCA
GTCCGCCCTGAGCAAAGACCCCAACGAGAAGCGCGATCACATGGTCCTGCTGGAGTTC
GTGACCGCCGCCGGGATCACTCTCGGCATGGACGAGCTGTACAAGTAAGGAGAAGAG
AAGGAAGAGAAAA
eGFP-U-depleted (maximum exchange)
(SEQ ID No: 28)
GGGATACGCCGCCACCATGGTGAGCAAGGGCGAGGAGCTGTTCACCGGGGTGGTGCC
CATCCTGGTCGAGCTGGACGGCGACGTAAACGGCCACAAGTTCAGCGTGTCCGGCGA
GGGCGAGGGCGACGCCACCTACGGCAAGCTGACCCTGAAGTTCATCTGCACCACCGG
CAAGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCCCG
AAGGCTACGTCCAGGAGCGCACCATCTCCTTCAAGGACGACGGCAACTACAAGACCC
GCGCCGAGGTGAAGTTCGAGGGCGACACCCTGGTGAACCGCATCGAGCTGAAGGGCA
TCGACTTCAAGGAGGACGGCAACATCCTGGGGCACAAGCTGGAGTACAACTACAACA
GCCACAACGTCTACATCATGGCCGACAAGCAGAAGAACGGCATCAAGGCCAACTTCA
AGATCCGCCACAACATCGAGGACGGCAGCGTGCAGCTCGCCGACCACTACCAGCAGA
ACACCCCCATCGGCGACGGCCCCGTGCTGCTGCCCGACAACCACTACCTGAGCACCCA
GTCCGCCCTGAGCAAAGACCCCAACGAGAAGCGCGACCACATGGTCCTGCTGGAGTT
CGTGACCGCCGCCGGGATCACACTCGGCATGGACGAGCTGTACAAGTAACCACAACA
CAACCAACACAAAA
eGFP-UC-depleted (maximum exchange)
(SEQ ID No: 29)
GGGATACGCCGCCACCATGGTGAGCAAGGGGGAGGAGCTGTTCACAGGGGTGGTGCC
AATCCTGGTCGAGCTGGACGGGGACGTAAACGGGCACAAGTTCAGCGTGAGCGGGGA
GGGGGAGGGGGACGCAACATACGGGAAGCTGACACTGAAGTTCATCTGCACAACAGG
GAAGCTGCCAGTGCCATGGCCAACACTCGTGACAACACTGACATACGGGGTGCAGTG
CTTCAGCAGATACCCAGACCACATGAAGCAGCACGACTTCTTCAAGAGCGCAATGCCA
GAAGGGTACGTCCAGGAGAGAACAATCAGCTTCAAGGACGACGGGAACTACAAGACA
AGAGCAGAGGTGAAGTTCGAGGGGGACACACTGGTGAACAGAATCGAGCTGAAGGG
GATCGACTTCAAGGAGGACGGGAACATCCTGGGGCACAAGCTGGAGTACAACTACAA
CAGCCACAACGTCTACATCATGGCAGACAAGCAGAAGAACGGGATCAAGGCAAACTT
CAAGATCAGACACAACATCGAGGACGGGAGCGTGCAGCTCGCAGACCACTACCAGCA
GAACACACCAATCGGGGACGGGCCAGTGCTGCTGCCAGACAACCACTACCTGAGCAC
ACAGAGCGCACTGAGCAAAGACCCAAACGAGAAGAGAGACCACATGGTCCTGCTGGA
GTTCGTGACAGCAGCAGGGATCACTCTCGGGATGGACGAGCTGTACAAGTAAGGAGA
AGAGAAGGAAGAGAAAA
eGFP-UG-depleted (maximum exchange)
(SEQ ID No: 30)
GGGATACGCCGCCACCATGGTCAGCAAAGGCGAAGAACTCTTCACCGGAGTCGTCCC
CATCCTCGTCGAACTCGACGGCGACGTAAACGGCCACAAATTCAGCGTCTCCGGCGAA
GGCGAAGGCGACGCCACCTACGGCAAACTCACCCTCAAATTCATCTGCACCACCGGCA
AACTCCCCGTCCCCTGGCCCACCCTCGTCACCACCCTCACCTACGGCGTCCAATGCTTC
AGCCGCTACCCCGACCACATGAAACAACACGACTTCTTCAAATCCGCCATGCCCGAAG
GCTACGTCCAAGAACGCACCATCTCCTTCAAAGACGACGGCAACTACAAAACCCGCG
CCGAAGTCAAATTCGAAGGCGACACCCTCGTCAACCGCATCGAACTCAAAGGCATCG
ACTTCAAAGAAGACGGCAACATCCTCGGACACAAACTCGAATACAACTACAACAGCC
ACAACGTCTACATCATGGCCGACAAACAAAAAAACGGCATCAAAGCCAACTTCAAAA
TCCGCCACAACATCGAAGACGGCAGCGTCCAACTCGCCGACCACTACCAACAAAACA
CCCCCATCGGCGACGGCCCCGTCCTCCTCCCCGACAACCACTACCTCAGCACCCAATC
CGCCCTCAGCAAAGACCCCAACGAAAAACGCGACCACATGGTCCTCCTCGAATTCGTC
ACCGCCGCCGGAATCACACTCGGCATGGACGAACTCTACAAATAACCACAACACAAC
CAACACAAAA
eGFP- C-depleted (maximum exchange of only C-containing but not U-
containing codons)
(SEQ ID No: 31)
GGGATACGCCGCCACCATGGTGAGCAAGGGAGAGGAGCTGTTCACAGGGGTGGTGCC
AATCCTGGTCGAGCTGGACGGAGACGTAAACGGACACAAGTTCAGCGTGTCCGGAGA
GGGAGAGGGAGATGCAACATACGGAAAGCTGACACTGAAGTTCATCTGCACAACAGG
AAAGCTGCCAGTGCCATGGCCAACACTCGTGACAACACTGACATACGGAGTGCAGTG
CTTCAGCCGGTACCCAGACCACATGAAGCAGCACGACTTCTTCAAGTCCGCAATGCCA
GAAGGATACGTCCAGGAGCGGACAATCTCCTTCAAGGACGACGGAAACTACAAGACA
CGGGCAGAGGTGAAGTTCGAGGGAGACACACTGGTGAACCGGATCGAGCTGAAGGGA
ATCGACTTCAAGGAGGACGGAAACATCCTGGGGCACAAGCTGGAGTACAACTACAAC
AGCCACAACGTCTATATCATGGCAGACAAGCAGAAGAACGGAATCAAGGCAAACTTC
AAGATCCGGCACAACATCGAGGACGGAAGCGTGCAGCTCGCAGACCACTACCAGCAG
AACACACCAATCGGAGACGGACCAGTGCTGCTGCCAGACAACCACTACCTGAGCACA
CAGTCCGCACTGAGCAAAGACCCAAACGAGAAGCGGGATCACATGGTCCTGCTGGAG
TTCGTGACAGCAGCAGGGATCACTCTCGGAATGGACGAGCTGTACAAGTAAGGAGAA
GAGAAGGAAGAGAAAA
eGFP-C2-depleted (maximum exchange of all C-containing codons)
(SEQ ID No: 32)
GGGATACGCCGCCACCATGGTGAGCAAGGGAGAGGAGCTGTTCACAGGGGTGGTGCC
AATACTGGTGGAGCTGGACGGAGACGTAAACGGACACAAGTTCAGCGTGAGTGGAGA
GGGAGAGGGAGATGCAACATACGGAAAGCTGACACTGAAGTTCATATGCACCACAGG
AAAGCTGCCAGTGCCATGGCCAACACTGGTGACAACACTGACATACGGAGTGCAGTG
CTTCAGCCGGTACCCAGACCACATGAAGCAGCACGACTTCTTCAAGAGTGCAATGCCA
GAAGGATACGTGCAGGAGCGGACAATAAGTTTCAAGGACGACGGAAACTACAAGACA
CGGGCAGAGGTGAAGTTCGAGGGAGACACACTGGTGAACCGGATAGAGCTGAAGGG
AATAGACTTCAAGGAGGACGGAAACATACTGGGGCACAAGCTGGAGTACAACTACAA
CAGCCACAACGTGTATATAATGGCAGACAAGCAGAAGAACGGAATAAAGGCAAACTT
CAAGATACGGCACAACATAGAGGACGGAAGCGTGCAGCTGGCAGACCACTACCAGCA
GAACACACCAATAGGAGACGGACCAGTGCTGCTGCCAGACAACCACTACCTGAGCAC
ACAGAGTGCACTGAGCAAAGACCCAAACGAGAAGCGGGATCACATGGTGCTGCTGGA
GTTCGTGACAGCAGCAGGGATAACTCTGGGAATGGACGAGCTGTACAAGTAAGGAGA
AGAGAAGGAAGAGAAAA
mEPO-WT
(SEQ ID No: 33)
GGGAAACTGCCAAGATGGGGGTGCCCGAACGTCCCACCCTGCTGCTTTTACTCTCCTT
GCTACTGATTCCTCTGGGCCTCCCAGTCCTCTGTGCTCCCCCACGCCTCATCTGCGACA
GTCGAGTTCTGGAGAGGTACATCTTAGAGGCCAAGGAGGCAGAAAATGTCACGATGG
GTTGTGCAGAAGGTCCCAGACTGAGTGAAAATATTACAGTCCCAGATACCAAAGTCA
ACTTCTATGCTTGGAAAAGAATGGAGGTGGAAGAACAGGCCATAGAAGTTTGGCAAG
GCCTGTCCCTGCTCTCAGAAGCCATCCTGCAGGCCCAGGCCCTGCTAGCCAATTCCTCC
CAGCCACCAGAGACCCTTCAGCTTCATATAGACAAAGCCATCAGTGGTCTACGTAGCC
TCACTTCACTGCTTCGGGTACTGGGAGCTCAGAAGGAATTGATGTCGCCTCCAGATAC
CACCCCACCTGCTCCACTCCGAACACTCACAGTGGATACTTTCTGCAAGCTCTTCCGGG
TCTACGCCAACTTCCTCCGGGGGAAACTGAAGCTGTACACGGGAGAGGTCTGCAGGA
GAGGGGACAGGTGAGGAGAAGAGAAGGAAGAGAAAA
mEPO-U-depleted (maximum exchange)
(SEQ ID No: 34)
GGGAAACTGCCAAGATGGGGGTGCCCGAACGACCCACCCTGCTGCTCTTACTCTCCCT
CCTACTGATCCCCCTGGGCCTCCCAGTCCTCTGCGCACCCCCACGCCTCATCTGCGACA
GCCGAGTCCTGGAGAGGTACATCTTAGAGGCCAAGGAGGCAGAAAACGTCACGATGG
GATGCGCAGAAGGACCCAGACTGAGCGAAAACATCACAGTCCCAGACACCAAAGTCA
ACTTCTACGCATGGAAAAGAATGGAGGTGGAAGAACAGGCCATAGAAGTCTGGCAAG
GCCTGTCCCTGCTCTCAGAAGCCATCCTGCAGGCCCAGGCCCTGCTAGCCAACTCCTC
CCAGCCACCAGAGACCCTCCAGCTCCACATAGACAAAGCCATCAGCGGACTACGAAG
CCTCACATCACTGCTCCGGGTACTGGGAGCACAGAAGGAACTCATGTCGCCCCCAGAC
ACCACCCCACCCGCACCACTCCGAACACTCACAGTGGACACATTCTGCAAGCTCTTCC
GGGTCTACGCCAACTTCCTCCGGGGGAAACTGAAGCTGTACACGGGAGAGGTCTGCA
GGAGAGGGGACAGGTAACCACAACACAACCAACACAAAA
mEPO-UC-depleted (maximum exchange)
(SEQ ID No: 35)
GGGAAACTGCCAAGATGGGGGTGCCAGAACGACCAACACTGCTGCTCCTACTCAGCTT
GCTACTGATCCCACTGGGGCTCCCAGTCCTCTGCGCACCACCAAGACTCATCTGCGAC
AGCCGAGTACTGGAGAGGTACATCCTAGAGGCAAAGGAGGCAGAAAACGTCACGATG
GGATGCGCAGAAGGACCAAGACTGAGCGAAAACATCACAGTCCCAGACACAAAAGTC
AACTTCTACGCATGGAAAAGAATGGAGGTGGAAGAACAGGCAATAGAAGTATGGCAA
GGGCTGAGCCTGCTCAGCGAAGCAATCCTGCAGGCACAGGCACTGCTAGCAAACAGC
AGCCAGCCACCAGAGACACTCCAGCTCCACATAGACAAAGCAATCAGCGGACTACGA
AGCCTCACTAGCCTGCTCAGGGTACTGGGAGCACAGAAGGAATTGATGTCGCCACCA
GACACAACACCACCAGCACCACTCCGAACACTCACAGTGGACACTTTCTGCAAGCTCT
TCAGGGTCTACGCAAACTTCCTCAGGGGGAAACTGAAGCTGTACACGGGAGAGGTCT
GCAGGAGAGGGGACAGGTGAGGAGAAGAGAAGGAAGAGAAAA
mEPO-UG-depleted (maximum exchange)
(SEQ ID No: 36)
GGGAAACTGCCAAGATGGGAGTCCCCGAACGACCCACCCTCCTCCTCTTACTCTCCCT
CCTACTCATCCCACTCGGCCTCCCAGTCCTCTGCGCACCCCCACGCCTCATCTGCGACA
GCCGAGTCCTCGAAAGATACATCTTAGAAGCCAAAGAAGCAGAAAACGTCACAATGG
GATGCGCAGAAGGACCCAGACTCAGCGAAAACATCACAGTCCCAGACACCAAAGTCA
ACTTCTACGCATGGAAAAGAATGGAAGTCGAAGAACAAGCCATAGAAGTCTGGCAAG
GCCTCTCCCTCCTCTCAGAAGCCATCCTCCAAGCCCAAGCCCTCCTAGCCAACTCCTCC
CAACCACCAGAAACCCTCCAACTCCACATAGACAAAGCCATCAGCGGACTACGAAGC
CTCACATCACTCCTCCGCGTACTCGGAGCACAAAAAGAACTCATGTCACCACCAGACA
CCACCCCACCAGCACCACTCCGAACACTCACAGTCGACACATTCTGCAAACTCTTCCG
CGTCTACGCCAACTTCCTCCGCGGAAAACTCAAACTCTACACAGGAGAAGTCTGCAGA
AGAGGAGACAGATAACCACAACACAACCAACACAAAA
mEPO-C-depleted (maximum exchange of only C-containing but not U-
containing codons)
(SEQ ID No: 37)
GGGAAACTGCCAAGATGGGGGTGCCAGAACGTCCAACACTGCTGCTTTTACTCTCCTT
GCTACTGATTCCTCTGGGACTCCCAGTCCTCTGTGCTCCACCACGGCTCATCTGCGACA
GTCGAGTTCTGGAGAGGTACATCTTAGAGGCAAAGGAGGCAGAAAATGTCACGATGG
GTTGTGCAGAAGGTCCAAGACTGAGTGAAAATATTACAGTCCCAGATACAAAAGTCA
ACTTCTATGCTTGGAAAAGAATGGAGGTGGAAGAACAGGCAATAGAAGTTTGGCAAG
GACTGTCCCTGCTCTCAGAAGCAATCCTGCAGGCACAGGCACTGCTAGCAAATTCCTC
CCAGCCACCAGAGACACTTCAGCTTCATATAGACAAAGCAATCAGTGGTCTACGTAGC
CTCACTTCACTGCTTCGGGTACTGGGAGCTCAGAAGGAATTGATGTCGCCTCCAGATA
CAACACCACCTGCTCCACTCCGAACACTCACAGTGGATACTTTCTGCAAGCTCTTCCG
GGTCTACGCAAACTTCCTCCGGGGGAAACTGAAGCTGTACACGGGAGAGGTCTGCAG
GAGAGGGGACAGGTGAGGAGAAGAGAAGGAAGAGAAAA
mEPO-C2-depleted (maximum exchange of all C-containing codons)
(SEQ ID No: 38)
GGGAAACTGCCAAGATGGGGGTGCCAGAACGTCCAACACTGCTGCTTTTACTGAGTTT
GCTACTGATTCCTCTGGGACTGCCAGTGCTGTGTGCTCCACCACGGCTGATATGCGAC
AGTCGAGTTCTGGAGAGGTACATATTAGAGGCAAAGGAGGCAGAAAATGTGACGATG
GGTTGTGCAGAAGGTCCAAGACTGAGTGAAAATATTACAGTGCCAGATACAAAAGTG
AACTTCTATGCTTGGAAAAGAATGGAGGTGGAAGAACAGGCAATAGAAGTTTGGCAA
GGACTGAGTCTGCTGAGTGAAGCAATACTGCAGGCACAGGCACTGCTAGCAAATAGT
AGTCAGCCACCAGAGACACTTCAGCTTCATATAGACAAAGCAATAAGTGGTCTACGTA
GCCTGACTAGTCTGCTTCGGGTACTGGGAGCTCAGAAGGAATTGATGAGTCCTCCAGA
TACAACACCACCTGCTCCACTGCGAACACTGACAGTGGATACTTTCTGCAAGCTGTTC
CGGGTGTACGCAAACTTCCTGCGGGGGAAACTGAAGCTGTACACGGGAGAGGTGTGC
AGGAGAGGGGACAGGTGAGGAGAAGAGAAGGAAGAGAAAA

### CODON EXCHANGE TABLES

The following codon exchange tables are used by the algorithm to generate a new coding sequence for the messenger RNA with the desired base-usage. The tables are to be used as examples only; any combination might be used that leads to the general effect of reducing the Cytidine or Uridine and Cytidine content of the messenger RNA.
Cytidine-depletion without the intention to reduce Uridine, although this might happen to a minor extent. Also, the exchange aims to respect codon usage frequency, by exchanging high frequency codons with high frequency codons, and exchange low frequency codons with low frequency codons.

Codon exchange table 1A

| | | | | Freq human | Freq human | |
|---|---|---|---|---|---|---|
| | Original | Swap | | codon usage | codon usage | Amino |
| | codon | codon | Direction | (original) | (swap) | acid |
| 1 | CTT | TTG | uni | 13.2 | 12.9 | L |
| 2 | CTC | CTG | uni | 19.6 | 39.6 | L |
| 3 | ATC | ATA | uni | 20.8 | 7.5 | I* |
| 4 | GTC | GTG | uni | 14.5 | 28.1 | V* |
| 5 | TCT | AGT | uni | 15.2 | 12.1 | S |
| 6 | TCC | AGT | uni | 17.7 | 12.1 | S |
| 7 | TCA | AGT | uni | 12.2 | 12.1 | S |
| 8 | TCG | AGT | uni | 4.4 | 12.1 | S* |
| 9 | CCC | CCA | uni | 19.8 | 16.9 | P |
| 10 | ACC | ACA | uni | 18.9 | 15.1 | T |
| 11 | GCC | GCA | uni | 27.7 | 15.8 | A* |
| 12 | CGC | CGG | uni | 10.4 | 11.4 | R |
| 13 | GGC | GGA | uni | 22.2 | 16.5 | G |

*Reduced frequency
Note:
Rules are to change every C to A or G or U. Reducing C takes precedent on codon frequency.

Cytidine-depletion without the intention to reduce Uridine, although this might happen to a minor extent. The reduction of cytidine is combined with exchanging the codon for the highest frequency codon available. This is also applied to codons that would not be changed because of Cytidine content. Cytidine reduction takes precedent over codon frequency optimization.

Codon exchange table 1B

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTA | TTG | uni | 7.7 | 12.9 | L |
| 2 | CTT | CTG | uni | 13.2 | 39.6 | L |
| 3 | CTC | CTG | uni | 19.6 | 39.6 | L |
| 4 | CTA | CTG | uni | 7.2 | 39.6 | L |
| 5 | ATC | ATA | uni | 20.8 | 7.5 | I |
| 6 | GTT | GTG | uni | 11 | 28.1 | V |
| 7 | GTC | GTG | uni | 14.5 | 28.1 | V |
| 8 | GTA | GTG | uni | 7.1 | 28.1 | V |
| 9 | TCT | AGC | uni | 15.2 | 19.5 | S |
| 10 | TCC | AGC | uni | 17.7 | 19.5 | S |
| 11 | TCA | AGC | uni | 12.2 | 19.5 | S |
| 12 | TCG | AGC | uni | 4.4 | 19.5 | S |
| 13 | CCC | CCA | uni | 19.8 | 16.9 | P |
| 14 | CCG | CCA | uni | 6.9 | 16.9 | P |
| 15 | ACT | ACA | uni | 13.1 | 15.1 | T |
| 16 | ACC | ACA | uni | 18.1 | 15.1 | T |
| 17 | ACG | ACA | uni | 6.1 | 15.1 | T |
| 18 | CAA | CAG | uni | 12.3 | 34.2 | Q |
| 19 | AAA | AAG | uni | 24.4 | 31.9 | K |
| 20 | GAA | GAG | uni | 29 | 39.6 | E |
| 21 | CGT | AGA | uni | 4.5 | 12.2 | R |
| 22 | CGC | AGA | uni | 10.4 | 12.2 | R |
| 23 | CGA | AGA | uni | 6.2 | 12.2 | R |
| 24 | CGG | AGA | uni | 11.4 | 12.2 | R |
| 25 | AGG | AGA | uni | 12 | 12.2 | R |
| 26 | GGT | GGA | uni | 10.8 | 16.5 | G |
| 27 | GGC | GGA | uni | 22.2 | 16.5 | G |
| 28 | GGG | GGA | uni | 16.5 | 16.5 | G |

Note:
Rules are: change every C to A or G but not U. Reducing C takes precedent on codon frequency.
If high codon frequency requires introduction of C or U, then take another lower codon frequency or don't change.

Cytidine-depletion combined with Uridine-depletion, with Cytidine taking precedent over Uridine. Also, the exchange aims to respect codon usage frequency, by exchanging high frequency codons with high frequency codons, and exchange low frequency codons with low frequency codons.

Codon exchange table 2A

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTC | TTT | uni | 20.3 | 17.6 | F |
| 2 | CTA | TTA | uni | 7.2 | 7.7 | L |
| 3 | CTT | TTG | uni | 13.2 | 12.9 | L |
| 4 | ATT | ATA | uni | 16 | 7.5 | I |
| 5 | ATC | ATA | uni | 20.8 | 7.5 | I |
| 6 | GTT | GTA | uni | 11 | 7.1 | V |
| 7 | GTC | GTG | uni | 14.5 | 28.1 | V |
| 8 | TCT | AGC | uni | 15.2 | 19.5 | S |
| 9 | TCC | AGC | uni | 17.7 | 19.5 | S |
| 10 | TCA | AGC | uni | 12.2 | 19.5 | S |
| 11 | CCT | CCA | uni | 17.5 | 16.9 | P |
| 12 | CCC | CCA | uni | 19.8 | 16.9 | P |
| 13 | ACT | ACA | uni | 13.1 | 15.1 | T |
| 14 | ACC | ACA | uni | 18.9 | 15.1 | T |
| 15 | GCT | GCA | uni | 18.4 | 15.8 | A |
| 16 | GCC | GCA | uni | 27.7 | 15.8 | A |
| 17 | TAC | TAT | uni | 15.3 | 12.2 | Y |
| 18 | CAC | CAT | uni | 15.1 | 10.9 | H |
| 19 | AAC | AAT | uni | 19.1 | 17 | N |
| 20 | GAC | GAT | uni | 25.1 | 21.8 | D |
| 21 | TGC | TGT | uni | 12.6 | 10.6 | C |
| 22 | CGT | CGA | uni | 4.5 | 6.2 | R |
| 23 | CGC | AGA | uni | 10.4 | 12.2 | R |
| 24 | AGC | AGT | uni | 19.5 | 12.1 | S |
| 25 | CGG | AGG | uni | 11.4 | 12 | R |
| 26 | GGT | GGA | uni | 10.8 | 16.5 | G |
| 27 | GGC | GGG | uni | 22.2 | 16.5 | G |

Note:
C-depletion has precedent on U-depletion

Cytidine-depletion combined with Uridine-depletion, with Cytidine taking precedent over Uridine. The reduction of cytidine is combined with exchanging the codon for the highest frequency codon available. This is also applied to codons that would not be changed because of Cytidine or Uridine content. Cytidine or Uridine reduction takes precedent over codon frequency optimization.

Codon exchange table 2B

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTC | TTT | uni | 20.3 | 17.6 | F |
| 2 | TTA | TTG | uni | 7.7 | 12.9 | L |
| 3 | CTT | CTG | uni | 13.2 | 39.6 | L |
| 4 | CTC | CTG | uni | 19.6 | 39.6 | L |
| 5 | CTA | CTG | uni | 7.2 | 39.6 | L |
| 6 | ATT | ATA | uni | 16 | 7.5 | I* |
| 7 | ATC | ATA | uni | 20.8 | 7.5 | I* |
| 8 | GTT | GTG | uni | 11 | 28.1 | V |
| 9 | GTC | GTG | uni | 14.5 | 28.1 | V |
| 10 | GTA | GTG | uni | 7.1 | 28.1 | V |
| 11 | TCT | AGT | uni | 15.2 | 12.1 | S |
| 12 | TCC | AGT | uni | 17.7 | 12.1 | S |
| 13 | TCA | AGT | uni | 12.2 | 12.1 | S |
| 14 | TCG | AGT | uni | 4.4 | 12.1 | S |
| 15 | CCT | CCA | uni | 17.5 | 16.9 | P |
| 16 | CCC | CCA | uni | 19.8 | 16.9 | P |
| 17 | CCG | CCA | uni | 6.9 | 16.9 | P |
| 18 | ACT | ACA | uni | 13.1 | 15.1 | T |
| 19 | ACC | ACA | uni | 18.9 | 15.1 | T |
| 20 | ACG | ACA | uni | 6.1 | 15.1 | T |
| 21 | GCT | GCA | uni | 18.4 | 15.8 | A |
| 22 | GCC | GCA | uni | 27.7 | 15.8 | A* |
| 23 | GCG | GCA | uni | 7.4 | 15.8 | A |
| 24 | TAC | TAT | uni | 15.3 | 12.2 | Y |
| 25 | CAC | CAT | uni | 15.1 | 10.9 | H |
| 26 | CAA | CAG | uni | 12.3 | 34.2 | Q |
| 27 | AAC | AAT | uni | 19.1 | 17 | N |
| 28 | AAA | AAG | uni | 24.4 | 31.9 | K |
| 29 | GAC | GAT | uni | 25.1 | 21.8 | D |
| 30 | GAA | GAG | uni | 29 | 39.6 | E |
| 31 | TGC | TGT | uni | 12.6 | 10.6 | C |
| 32 | CGT | AGA | uni | 4.5 | 12.2 | R |
| 33 | CGC | AGA | uni | 10.4 | 12.2 | R |
| 34 | CGA | AGA | uni | 6.2 | 12.2 | R |
| 35 | CGG | AGA | uni | 11.4 | 12.2 | R |
| 36 | AGG | AGA | uni | 12 | 12.2 | R |
| 37 | AGC | AGT | uni | 19.5 | 12.1 | S |
| 38 | GGT | GGA | uni | 10.8 | 16.5 | G |
| 39 | GGC | GGA | uni | 22.2 | 16.5 | G |
| 40 | GGG | GGA | uni | 16.5 | 16.5 | G |

*frequency reduction
Note:
C-depletion has precedent on U-depletion

Cytidine-depletion combined with Uridine-depletion, with Uridine taking precedent over Cytidine. Also, the exchange aims to respect codon usage frequency, by exchanging high frequency codons with high frequency codons, and exchange low frequency codons with low frequency codons.

Codon exchange table 2C

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTT | TTC | uni | 17.6 | 20.3 | F |
| 2 | TTA | CTA | uni | 7.7 | 7.2 | L |
| 3 | CTT | CTC | uni | 13.2 | 19.6 | L |
| 4 | ATT | ATC | uni | 16 | 20.8 | I |
| 5 | GTT | GTA | uni | 11 | 7.1 | V |
| 6 | TCT | AGC | uni | 15.2 | 19.5 | S |
| 7 | TCC | AGC | uni | 17.7 | 19.5 | S |
| 8 | TCA | AGC | uni | 12.2 | 19.5 | S |
| 9 | CCT | CCA | uni | 17.5 | 16.9 | P |
| 10 | CCC | CCA | uni | 19.8 | 16.9 | P |
| 11 | ACT | ACA | uni | 13.1 | 15.1 | T |
| 12 | ACC | ACA | uni | 18.9 | 15.1 | T |
| 13 | GCT | GCA | uni | 18.4 | 15.8 | A |
| 14 | GCC | GCA | uni | 27.7 | 15.8 | A |
| 15 | TAT | TAC | uni | 12.2 | 15.3 | Y |
| 16 | CAT | CAC | uni | 10.9 | 15.1 | H |
| 17 | AAT | AAC | uni | 17 | 19.1 | N |
| 18 | GAT | GAC | uni | 21.8 | 25.1 | D |
| 19 | TGT | TGC | uni | 10.6 | 12.6 | C |
| 20 | CGT | CGA | uni | 4.5 | 6.2 | R |
| 21 | CGC | AGA | uni | 10.4 | 12.2 | R |
| 22 | AGT | AGC | uni | 12.1 | 19.5 | S |
| 23 | CGG | AGG | uni | 11.4 | 12 | R |
| 24 | GGT | GGA | uni | 10.8 | 16.5 | G |
| 25 | GGC | GGG | uni | 22.2 | 16.5 | G |

Cytidine-depletion combined with Uridine-depletion, with Uridine taking precedent over Cytidine. The reduction of cytidine is combined with exchanging the codon for the highest frequency codon available. This is also applied to codons that would not be changed because of Cytidine or Uridine content. Cytidine and Uridine reduction takes precedent over codon frequency optimization.

Codon exchange table 2D

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTT | TTC | uni | 17.6 | 20.3 | F |
| 2 | TTA | CTG | uni | 7.7 | 39.6 | L |
| 3 | TTG | CTG | uni | 12.9 | 39.6 | L |
| 4 | CTT | CTG | uni | 13.2 | 39.6 | L |
| 5 | CTC | CTG | uni | 19.6 | 39.6 | L |
| 6 | CTA | CTG | uni | 7.2 | 39.6 | L |
| 7 | ATT | ATC | uni | 16 | 20.8 | I |
| 8 | GTT | GTG | uni | 11 | 28.1 | V |
| 9 | GTC | GTG | uni | 14.5 | 28.1 | V |
| 10 | GTA | GTG | uni | 7.1 | 28.1 | V |
| 11 | TCT | AGC | uni | 15.2 | 19.5 | S |
| 12 | TCC | AGC | uni | 17.7 | 19.5 | S |
| 13 | TCA | AGC | uni | 12.2 | 19.5 | S |
| 14 | TCG | AGC | uni | 4.4 | 19.5 | S |
| 15 | AGT | AGC | uni | 12.1 | 19.5 | S |
| 16 | CCT | CCA | uni | 17.5 | 16.9 | P |
| 17 | ccc | CCA | uni | 19.8 | 16.9 | P |
| 18 | CCG | CCA | uni | 6.9 | 16.9 | P |
| 19 | ACT | ACA | uni | 13.1 | 15.1 | T |
| 20 | ACC | ACA | uni | 18.9 | 15.1 | T |
| 21 | ACG | ACA | uni | 6.1 | 15.1 | T |
| 22 | GCT | GCA | uni | 18.4 | 15.8 | A |
| 23 | GCC | GCA | uni | 27.7 | 15.8 | A |
| 24 | GCG | GCA | uni | 7.4 | 15.8 | A |
| 25 | TAT | TAC | uni | 12.2 | 15.3 | Y |
| 26 | CAT | CAC | uni | 10.9 | 15.1 | H |
| 27 | CAA | CAG | uni | 12.3 | 34.2 | Q |
| 28 | AAT | AAC | uni | 17 | 19.1 | N |
| 29 | AAA | AAG | uni | 24.4 | 31.9 | K |
| 30 | GAT | GAC | uni | 21.8 | 25.1 | D |
| 31 | GAA | GAG | uni | 29 | 39.6 | E |
| 32 | TGT | TGC | uni | 10.6 | 12.6 | C |
| 33 | CGT | AGA | uni | 4.5 | 12.2 | R |
| 34 | CGC | AGA | uni | 10.4 | 12.2 | R |
| 35 | CGA | AGA | uni | 6.2 | 12.2 | R |
| 36 | CGG | AGA | uni | 11.4 | 12.2 | R |
| 37 | AGG | AGA | uni | 12 | 12.2 | R |
| 38 | GGT | GGA | uni | 10.8 | 16.5 | G |
| 39 | GGC | GGA | uni | 22.2 | 16.5 | G |

Note:
U-depletion takes precedent over C-depletion, which takes precedent over codon optimality.

Guanosine-depletion combined with Uridine-depletion, with Uridine taking precedent over Guanosine. Also, the exchange aims to respect codon usage frequency, by exchanging high frequency codons with high frequency codons, and exchange low frequency codons with low frequency codons.

Codon exchange table 3

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTT | TTC | uni | 0.45 | 0.55 | F |
| 2 | TTA | CTA | uni | 0.07 | 0.07 | L |
| 3 | TTG | CTT | uni | 0.13 | 0.13 | L |
| 4 | ATT | ATC | uni | 0.36 | 0.48 | I |
| 5 | GTT | GTA | uni | 0.18 | 0.11 | V |
| 6 | GTA | GTC | uni | 0.11 | 0.24 | V |
| 7 | TCT | AGC | uni | 0.18 | 0.24 | S |
| 8 | TCA | AGC | uni | 0.15 | 0.24 | S |
| 9 | TCC | AGC | uni | 0.22 | 0.24 | S |
| 10 | AGT | AGC | uni | 0.15 | 0.24 | S |
| 11 | CCT | CCA | uni | 0.28 | 0.27 | P |
| 12 | CCC | CCA | uni | 0.33 | 0.27 | P |
| 13 | ACT | ACA | uni | 0.24 | 0.28 | T |
| 14 | TAT | TAC | uni | 0.43 | 0.57 | Y |
| 15 | CAT | CAC | uni | 0.41 | 0.59 | H |
| 16 | AAT | AAC | uni | 0.46 | 0.54 | N |
| 17 | AAG | AAA | uni | 0.58 | 0.42 | K |
| 18 | GAT | GAC | uni | 0.46 | 0.54 | D |
| 19 | CGT | CGA | uni | 0.08 | 0.11 | R |
| 20 | CGG | CGC | uni | 0.21 | 0.19 | R |
| 21 | CGG | CGC | uni | 0.21 | 0.19 | R |
| 22 | AGG | AGA | uni | 0.2 | 0.2 | R |
| 23 | AGA | CGC | uni | 0.2 | 0.19 | R |

Uridine-depletion without the intention to reduce any other nucleotide, although this might happen to a minor extent. Also, the exchange aims to respect codon usage frequency, by exchanging high frequency codons with high frequency codons, and exchange low frequency codons with low frequency codons.

Codon exchange table 4A

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTT | TTC | uni | 17.6 | 20.3 | F |
| 2 | TTG | CTC | uni | 12.9 | 19.6 | L |
| 3 | CTT | CTC | uni | 13.2 | 19.6 | L |
| 4 | ATT | ATC | uni | 16 | 20.8 | I |
| 5 | GTT | GTC | uni | 11 | 14.5 | V |
| 6 | GCT | GCA | uni | 18.4 | 15.8 | A |
| 7 | ACT | ACA | uni | 13.1 | 15.1 | T |
| 8 | CCT | CCC | uni | 17.5 | 19.8 | P |
| 9 | TCT | TCC | uni | 15.2 | 17.7 | S |
| 10 | TAT | TAC | uni | 12.2 | 15.3 | Y |
| 11 | CAT | CAC | uni | 10.9 | 15.1 | H |
| 12 | AAT | AAC | uni | 17 | 19.1 | N |
| 13 | GAT | GAC | uni | 21.8 | 25.1 | D |
| 14 | TGT | TGC | uni | 10.6 | 12.6 | C |
| 15 | CGT | CGA | uni | 4.5 | 6.2 | R |
| 16 | AGT | AGC | uni | 12.1 | 19.5 | S |
| 17 | GGT | GGA | uni | 10.8 | 16.5 | G |

Uridine-depletion without the intention to reduce any other nucleotide, although this might happen to a minor extent. The reduction of uridine is combined with exchanging the codon for the highest frequency codon available. This is also applied to codons that would not be changed because of Uridine content. Uridine reduction takes precedent over codon frequency optimization.

Codon exchange table 4B

| | | | | Freq human | Freq human | |
|---|---|---|---|---|---|---|
| | Original | Swap | | codon usage | codon usage | Amino |
| | codon | codon | Direction | (original) | (swap) | acid |
| 1 | TTT | TTC | uni | 17.6 | 20.3 | F |
| 2 | TTA | CTG | uni | 7.7 | 39.6 | L** |
| 3 | TTG | CTG | uni | 12.9 | 39.6 | L |
| 4 | CTT | CTG | uni | 13.2 | 39.6 | L |
| 5 | CTC | CTG | uni | 19.6 | 39.6 | L** |
| 6 | CTA | CTG | uni | 7.2 | 39.6 | L |
| 7 | ATT | ATC | uni | 16 | 20.8 | I |
| 8 | ATA | ATC | uni | 7.5 | 20.8 | I |
| 9 | GTT | GTG | uni | 11 | 28.1 | V |
| 10 | GTC | GTG | uni | 14.5 | 28.1 | V** |
| 11 | GTA | GTG | uni | 7.1 | 28.1 | V |
| 12 | TCT | AGC | uni | 15.2 | 19.5 | S |
| 13 | TCC | AGC | uni | 17.7 | 19.5 | S** |
| 14 | TCA | AGC | uni | 12.2 | 19.5 | S |
| 15 | TCG | AGC | uni | 4.4 | 19.5 | S |
| 16 | AGT | AGC | uni | 12.1 | 19.5 | S |
| 17 | CCT | CCC | uni | 17.5 | 19.8 | P |
| 18 | CCG | CCC | uni | 6.9 | 19.8 | P |
| 19 | ACT | ACC | uni | 13.1 | 18.9 | T |
| 20 | ACG | ACC | uni | 6.1 | 18.9 | T |
| 21 | GCT | GCC | uni | 18.4 | 27.7 | A |
| 22 | GCA | GCC | uni | 15.8 | 27.7 | A |
| 23 | GCG | GCC | uni | 7.4 | 27.7 | A |
| 24 | TAT | TAC | uni | 12.2 | 15.3 | Y |
| 25 | CAT | CAC | uni | 10.9 | 15.1 | H |
| 26 | CAA | CAG | uni | 12.3 | 34.2 | Q |
| 27 | AAT | AAC | uni | 17 | 19.1 | N |
| 28 | AAA | AAG | uni | 24.4 | 31.9 | K |
| 29 | GAT | GAC | uni | 21.8 | 25.1 | D |
| 30 | GAA | GAG | uni | 29 | 39.6 | E |
| 31 | TGT | TGC | uni | 10.6 | 12.6 | C |
| 32 | CGT | AGA | uni | 4.5 | 12.2 | R |
| 33 | CGC | AGA | uni | 10.4 | 12.2 | R |
| 34 | CGA | AGA | uni | 6.2 | 12.2 | R |
| 35 | CGG | AGA | uni | 11.4 | 12.2 | R |
| 36 | AGG | AGA | uni | 12 | 12.2 | R |
| 37 | GGT | GGC | uni | 10.8 | 22.2 | G |
| 38 | GGG | GGC | uni | 16.5 | 22.2 | G |
| 38 | GGA | GGC | uni | 16.5 | 22.2 | G |

**C-depletion

Cytidine-depletion without the intention to reduce Uridine, although this might happen to a minor extent. Also, the exchange aims to respect codon usage frequency, by exchanging high frequency codons with high frequency codons, and exchange low frequency codons with low frequency codons. This codon exchange table is used to obtain a dose-effect of C-depletion for comparison to C2-depletion (codon exchange table 1A)

Codon exchange table 5

| | | | | Freq human | Freq human | |
|---|---|---|---|---|---|---|
| | Original | Swap | | codon usage | codon usage | Amino |
| | codon | codon | Direction | (original) | (swap) | acid |
| 9 | CCC | CCA | uni | 19.8 | 16.9 | P |
| 10 | ACC | ACA | uni | 18.9 | 15.1 | T |
| 11 | GCC | GCA | uni | 27.7 | 15.8 | A* |
| 12 | CGC | CGG | uni | 10.4 | 11.4 | R |
| 13 | GGC | GGA | uni | 22.2 | 16.5 | G |

*Reduced frequency
Note:
Rules are to change every C to A or Gor U. Reducing C takes precedent on codon frequency.

1-37. (canceled)

38. A method for decreasing the immunogenicity of an RNA molecule and/or at least maintaining the translation efficacy thereof, the method comprising:
a) providing a wildtype DNA sequence as a template for RNA transcription;
b) selecting from the DNA sequence the coding sequence of the sense DNA strand, which comprises the sequence from the ATG codon to the first in-frame stop codon;
c) dividing the coding sequence into codons;
d) exchanging one or more codons that comprise one or more cytidine nucleotides for an available alternative codon comprising less cytidine nucleotides and resulting in a similar amino acid to obtain a DNA molecule with a modified DNA sequence; and
e) producing a modified RNA molecule from the DNA molecule with the modified DNA sequence,
wherein the exchange of codons results in the total cytidine content of the modified RNA molecule being at least 10% less than the total cytidine content of the corresponding RNA molecule transcribed from said wildtype DNA sequence.

39. The method of claim 38, further comprising repeating step d) with codons comprising thymidine nucleotides before producing the modified RNA molecule, wherein the exchange of codons results in the total uridine content of the modified RNA molecule being at least 10% less than the total uridine content of the corresponding RNA molecule transcribed from said wild-type DNA sequence.

40. The method of claim 38, wherein the alternative codon encodes the same amino acid.

41. The method of claim 38, wherein the cytidine nucleotides and the thymidine nucleotides in a codon are replaced with another non-modified nucleotide, in particular a guanosine or adenosine nucleotide.

42. The method of claim 38, wherein codons are exchanged in a random fashion, or in the order of their appearance in the coding sequence.

43. The method of claim 38, wherein codons are exchanged with alternative codons that occur with the highest frequency in the human genome.

44. The method of claim 38, wherein the available alternative codon comprising less cytidine nucleotides encodes the same amino acid, or wherein the available alternative codon comprising less cytidine nucleotides result in conservative replacement of the encoded amino acid.

45. The method of claim 38, wherein the codons are exchanged according to any one of the codon exchange tables 1A, 1B, 2A, 2B, 2C, 2D.

46. An RNA molecule, which is modified as compared to a corresponding wildtype RNA molecule, wherein the modification comprises a reduction of cytidine nucleotides to the extent that the exchange of codons results in the total cytidine content being at least 10% less than the total cytidine content of the corresponding RNA molecule transcribed from said wild-type DNA sequence, wherein the modification optionally further comprises a reduction of uridine nucleotides to the extent that the exchange of codons results in the total uridine content being at least 10% less than the total uridine content of the corresponding RNA molecule transcribed from said wild-type DNA sequence, wherein the modified RNA molecule is in particular less immunogenic than the wildtype RNA molecule and/or upon translation results in a similar or higher protein production, in particular a significantly higher protein production than the wildtype RNA molecule.

47. The RNA molecule of claim 46, which is a long non-coding RNA or a messenger RNA molecule (mRNA) encoding a peptide, polypeptide or protein.

48. The RNA molecule of claim 46, wherein the nucleotides replacing the cytidines or uridines of the wild type RNA molecule in the modified RNA molecule are non-modified nucleotides, wherein the RNA molecule optionally comprises a modification as compared to a wildtype RNA molecule, which modification is a deletion and/or substitution of one or more of the cytidine nucleotides, in particular a substitution or deletion of one or more of the cytidine and optionally uridine nucleotides from an untranslated region of the RNA molecule.

49. The RNA molecule of claim 48, which is an mRNA and wherein the amino acid sequence of the peptide, polypeptide or protein encoded by the modified mRNA molecule is the same as the amino acid sequence of the polypeptide or protein encoded by the wildtype mRNA molecule, or is different from the amino acid sequence of the peptide, polypeptide or protein encoded by the wildtype mRNA molecule, which difference between the amino acid sequence encoded by the modified RNA sequence compared to the amino acid sequence encoded by the wildtype RNA sequence is less than 1/200 codons.

50. The RNA molecule of claim 46, wherein the RNA sequence is modified by substituting cytidine and optionally uridine nucleotides by adenosine or guanosine nucleotides, in particular non-modified adenosine or guanosine nucleotides.

51. The RNA molecule of claim 46, which is an mRNA and wherein the cytidine content and optionally the uridine content is reduced in the coding region of the mRNA, and/or wherein the cytidine content and optionally the uridine content is reduced in the non-coding region of the mRNA, in particular in the 5′UTR region and/or 3′UTR region.

52. The RNA molecule of claim 46, wherein in order of increased preference at least 10, 15, 20, 25, 30, 35, 40, 45, 50% of the cytidine and optionally uridine nucleotides of the RNA sequence of the wildtype RNA molecule are replaced by a nucleotide that is not cytidine or uridine, respectively, or deleted.

53. The RNA molecule of claim 48 for use in therapy, wherein the therapy is selected from replacement of absent and/or defective polypeptides or proteins having a biological activity, supplementation of an endogenous protein to enhance cellular processes counteracting a disorder or repress cellular processes causing a disorder, introduction of non-endogenous biologically active proteins in a patient,
wherein the therapy is in particular for treatment of disorders that involve inflammation, in particular chronic kidney disease, focal segmental glomerulosclerosis, lupus nephritis, glomerulonephritis, membranoproliferative glomerulonephritis, interstitial nephritis, IgA nephropathy (Berger's disease), pyelonephritis, Goodpasture's syndrome, Wegener's granulomatosis, acute kidney disease, kidney transplant rejection, inflammatory bowel disease, ulcerative colitis, Crohn's disease, coeliac disease, atopic dermatitis, psoriasis, eczema, Behçet's disease, acne, pyoderma, rosacea, systemic lupus erythematosus, asthma, chronic obstructive pulmonary disease, COPD, pneumonitis rheumatoid arthritis, periodontitis, sinusitis, transplant rejection, ischemia reperfusion injury (also known as reperfusion injury), atherosclerosis, vasculitis, inflammatory cornea disorders, diabetic nephropathy, sepsis, liver fibrosis/cirrhosis, or for use in diagnosis,
wherein the diagnosis is selected from detecting specific cells, detecting the presence or absence of proteins, in particular tumor suppressor proteins, proteins signaling inflammation, fibrosis and/or cell-stress, or for use in prophylaxis,
wherein the RNA molecule is used as a vaccine, in particular a vaccine against viruses, such as influenza viruses or corona viruses.

54. The RNA molecule of claim 46, obtainable or produced by a method comprising:
a) providing a wildtype DNA sequence as a template for RNA transcription;
b) selecting from the DNA sequence the coding sequence of the sense DNA strand, which comprises the sequence from the ATG codon to the first in-frame stop codon;
c) dividing the coding sequence into codons;
d) exchanging one or more codons that comprise one or more cytidine nucleotides for an available alternative codon comprising less cytidine nucleotides and resulting in a similar amino acid to obtain a DNA molecule with a modified DNA sequence; and
e) producing a modified RNA molecule from the DNA molecule with the modified DNA sequence,
wherein the exchange of codons results in the total cytidine content of the modified RNA molecule being at least 10% less than the total cytidine content of the corresponding RNA molecule transcribed from said wildtype DNA sequence.

55. A pharmaceutical composition comprising the modified RNA molecule of claim 46.

56. A use of the RNA molecule of claim 46 in genome editing, wherein the RNA molecule is for encoding an RNA-guided endonuclease and/or a guide RNA in CRISPR technology.


SEQUENCE LISTING (38 sequences)

SEQ ID NO: 1 - DNA, 17 bp, Artificial Sequence
  T7 promoter
taatacgactcactata

SEQ ID NO: 2 - DNA, 23 bp, Artificial Sequence
  Linear template Fwd primer
tacgtagcgctaatacgactcac

SEQ ID NO: 3 - DNA, 24 bp, Artificial Sequence
  Linear template Rvs primer
gtatcttatcatgtctgctcgaag

SEQ ID NO: 4 - DNA, 25 bp, Artificial Sequence
  UC-depleted and UG-depleted Fwd primer
ggaggtaatacgactcactataggg

SEQ ID NO: 5 - DNA, 23 bp, Artificial Sequence
  UC-depleted and UG-depleted Rvs primer
ttttgtgttggttgtgttgtggt

SEQ ID NO: 6 - DNA, 23 bp, Artificial Sequence
  UC-depleted and UG-depleted Rvs primer
ttttctcttccttctcttctcct

SEQ ID NO: 7 - DNA, 16 bp, Artificial Sequence
  5′UTR DNA template
gggaaacgccgccacc

SEQ ID NO: 8 - DNA, 22 bp, Artificial Sequence
  3′UTR DNA template
ggagaagagaaggaagagaaaa

SEQ ID NO: 9 - DNA, 22 bp, Artificial Sequence
  3′UTR DNA template
ccacaacacaaccaacacaaaa

SEQ ID NO: 10 - DNA, 14 bp, Artificial Sequence
  5′UTR DNA template
gggaaactgccaag

SEQ ID NO: 11 - DNA, 16 bp, Artificial Sequence
  5′UTR DNA template
gggatacgccgccacc

SEQ ID NO: 12 - DNA, 23 bp, Artificial Sequence
  Rvs primer DNA template
ttttctcttccttctcttctcct

SEQ ID NO: 13 - DNA, 23 bp, Artificial Sequence
  Rvs primer DNA template
ttttgtgttggttgtgttgtggt

SEQ ID NO: 14 - DNA, 600 bp, Artificial Sequence
  WT coding sequence secreted NanoLuc
atgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 15 - DNA, 579 bp, Mus musculus
  WT coding sequence murine Erythropoietin
atgggggtgcccgaacgtcccaccctgctgcttttactctccttgctactgattcctctgggcctcccagtcctctgtgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacaggtga

SEQ ID NO: 16 - DNA, 720 bp, Aequorea victoria
  WT coding sequence eGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctccttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 17 - protein, 192 aa, Mus musculus
  murine EPO protein
MGVPERPTLLLLLSLLLIPLGLPVLCAPPRLICDSRVLERYILEAKEAENVTMGCAEGPRLSENITVPDTKVNFYAWKRMEVEEQAIEVWQGLSLLSEAILQAQALLANSSQPPETLQLHIDKAISGLRSLTSLLRVLGAQKELMSPPDTTPPAPLRTLTVDTFCKLFRVYANFLRGKLKLYTGEVCRRGDR

SEQ ID NO: 18 - protein, 239 aa, Aequorea victoria
  eGFP protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTISFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 19 - protein, 199 aa, Artificial Sequence
  Nanoluciferase protein
MNSFSTSAFGPVAFSLGLLLVLPAAFPAPVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 20 - DNA, 672 bp, Artificial Sequence
  Secreted NanoLuc WT
gggatacgccgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggccgcgactctagagtcggggcggccggccgcttcgagcagacatgataagatac

SEQ ID NO: 21 - DNA, 638 bp, Artificial Sequence
  Secreted NanoLuc WT (control to other mRNAs)
gggaaacgccgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggagaagagaaggaagagaaaa

SEQ ID NO: 22 - DNA, 638 bp, Artificial Sequence
  Secreted NanoLuc U-depleted (maximum exchange)
gggaaacgccgccaccatgaactccttctccacaagcgccttcggaccagtcgccttctccctgggcctgctcctggtgctccccgcagccttccccgccccagtcttcacactcgaagacttcgtcggggactggcgacagacagccggctacaacctggaccaagtcctcgaacagggaggagtgtccagcctcttccagaacctcggggtgtccgtaactccgatccaaaggatcgtcctgagcggagaaaacgggctgaagatcgacatccacgtcatcatcccgtacgaaggactgagcggcgaccaaatgggccagatcgaaaaaatcttcaaggtggtgtaccccgtggacgaccaccacttcaaggtgatcctgcactacggcacactggtaatcgacggggtcacgccgaacatgatcgactacttcggacggccgtacgaaggcatcgccgtgttcgacggcaaaaagatcactgtaacagggaccctgtggaacggcaacaaaatcatcgacgagcgcctgatcaaccccgacggctccctgctgttccgagtaaccatcaacggagtgaccggctggcggctgtgcgaacgcatcctggcgtaaccacaacacaaccaacacaaaa

SEQ ID NO: 23 - DNA, 638 bp, Artificial Sequence
  Secreted NanoLuc UC-depleted (maximum exchange)
gggaaacgccgccaccatgaacagcttcagcacaagcgcattcggaccagtggcattcagcctgggactgctgctggtgctgccagcagcattcccagcaccagtcttcacactggaggacttcgtgggggactggagacagacagcaggatacaacctggaccaggtcctggagcagggaggagtgagcagcctgttccagaacctgggggtgagcgtgacaccaatccagagaatcgtcctgagcggagagaacgggctgaagatcgacatccacgtcatcatcccatacgagggactgagcggagaccagatgggacagatcgagaagatcttcaaggtggtgtacccagtggacgaccaccacttcaaggtgatcctgcactacggaacactggtgatcgacggggtgacaccaaacatgatcgactacttcggaagaccatacgagggaatcgcagtgttcgacggaaagaagatcacagtgacagggacactgtggaacggaaacaagatcatcgacgagagactgatcaacccagacggaagcctgctgttcagagtgacaatcaacggagtgacaggatggagactgtgcgagagaatcctggcataaggagaagagaaggaagagaaaa

SEQ ID NO: 24 - DNA, 638 bp, Artificial Sequence
  Secreted NanoLuc UG-depleted (maximum exchange)
gggaaacgccgccaccatgaactccttctccacaagcgccttcggaccagtcgccttctccctcggcctcctcctcgtcctcccagcagccttcccagccccagtcttcacactcgaagacttcgtcggagactggcgacaaacagccggctacaacctcgaccaagtcctcgaacaaggaggagtctccagcctcttccaaaacctcggagtctccgtaacaccaatccaaagaatcgtcctcagcggagaaaacggactcaaaatcgacatccacgtcatcatcccatacgaaggactcagcggcgaccaaatgggccaaatcgaaaaaatcttcaaagtcgtctacccagtcgacgaccaccacttcaaagtcatcctccactacggcacactcgtaatcgacggagtcacaccaaacatgatcgactacttcggacgcccatacgaaggcatcgccgtcttcgacggcaaaaaaatcacagtaacaggaaccctctggaacggcaacaaaatcatcgacgagcgcctcatcaaccccgacggctccctcctcttccgagtaaccatcaacggagtcaccggctggcgcctctgcgaacgcatcctcgcataaccacaacacaaccaacacaaaa

SEQ ID NO: 25 - DNA, 638 bp, Artificial Sequence
  Secreted NanoLuc C-depleted (maximum exchange of only C-containing but not U-containing codons)
gggaaacgccgccaccatgaactccttctccacaagcgcattcggtccagttgcattctccctgggactgctcctggtgttgcctgctgcattccctgcaccagtcttcacactcgaagatttcgttggggactggcgacagacagcaggatacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggagaccaaatgggacagatcgaaaaaatttttaaggtggtgtaccctgtggatgatcatcactttaaggtgatcctgcactatggaacactggtaatcgacggggttacgccgaacatgatcgactatttcggacggccgtatgaaggaatcgcagtgttcgacggaaaaaagatcactgtaacagggacactgtggaacggaaacaaaattatcgacgagcggctgatcaacccagacggatccctgctgttccgagtaacaatcaacggagtgacaggatggcggctgtgcgaacggattctggcgtaaggagaagagaaggaagagaaaa

SEQ ID NO: 26 - DNA, 638 bp, Artificial Sequence
  Secreted NanoLuc C2-depleted (maximum exchange of all C-containing codons)
gggaaacgccgccaccatgaacagtttcagtacaagcgcattcggtccagttgcattcagtctgggactgctgctggtgttgcctgctgcattccctgcaccagtgttcacactggaagatttcgttggggactggcgacagacagcaggatacaacctggaccaagtgcttgaacagggaggtgtgagtagtttgtttcagaatctgggggtgagtgtaactccgatacaaaggattgtgctgagcggtgaaaatgggctgaagatagacatacatgtgataataccgtatgaaggtctgagcggagaccaaatgggacagatagaaaaaatttttaaggtggtgtaccctgtggatgatcatcactttaaggtgatactgcactatggaacactggtaatagacggggttacgccgaacatgatagactatttcggacggccgtatgaaggaatagcagtgttcgacggaaaaaagataactgtaacagggacactgtggaacggaaacaaaattatagacgagcggctgataaacccagacggaagtctgctgttccgagtaacaataaacggagtgacaggatggcggctgtgcgaacggattctggcgtaaggagaagagaaggaagagaaaa

SEQ ID NO: 27 - DNA, 758 bp, Aequorea victoria
  eGFP WT
gggatacgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctccttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggagaagagaaggaagagaaaa

SEQ ID NO: 28 - DNA, 758 bp, Aequorea victoria
  eGFP U-depleted (maximum exchange)
gggatacgccgccaccatggtgagcaagggcgaggagctgttcaccggggtggtgcccatcctggtcgagctggacggcgacgtaaacggccacaagttcagcgtgtccggcgagggcgagggcgacgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctccttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctacatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgaccacatggtcctgctggagttcgtgaccgccgccgggatcacactcggcatggacgagctgtacaagtaaccacaacacaaccaacacaaaa

SEQ ID NO: 29 - DNA, 758 bp, Aequorea victoria
  eGFP UC-depleted (maximum exchange)
gggatacgccgccaccatggtgagcaagggggaggagctgttcacaggggtggtgccaatcctggtcgagctggacggggacgtaaacgggcacaagttcagcgtgagcggggagggggagggggacgcaacatacgggaagctgacactgaagttcatctgcacaacagggaagctgccagtgccatggccaacactcgtgacaacactgacatacggggtgcagtgcttcagcagatacccagaccacatgaagcagcacgacttcttcaagagcgcaatgccagaagggtacgtccaggagagaacaatcagcttcaaggacgacgggaactacaagacaagagcagaggtgaagttcgagggggacacactggtgaacagaatcgagctgaaggggatcgacttcaaggaggacgggaacatcctggggcacaagctggagtacaactacaacagccacaacgtctacatcatggcagacaagcagaagaacgggatcaaggcaaacttcaagatcagacacaacatcgaggacgggagcgtgcagctcgcagaccactaccagcagaacacaccaatcggggacgggccagtgctgctgccagacaaccactacctgagcacacagagcgcactgagcaaagacccaaacgagaagagagaccacatggtcctgctggagttcgtgacagcagcagggatcactctcgggatggacgagctgtacaagtaaggagaagagaaggaagagaaaa

SEQ ID NO: 30 - DNA, 758 bp, Aequorea victoria
  eGFP UG-depleted (maximum exchange)
gggatacgccgccaccatggtcagcaaaggcgaagaactcttcaccggagtcgtccccatcctcgtcgaactcgacggcgacgtaaacggccacaaattcagcgtctccggcgaaggcgaaggcgacgccacctacggcaaactcaccctcaaattcatctgcaccaccggcaaactccccgtcccctggcccaccctcgtcaccaccctcacctacggcgtccaatgcttcagccgctaccccgaccacatgaaacaacacgacttcttcaaatccgccatgcccgaaggctacgtccaagaacgcaccatctccttcaaagacgacggcaactacaaaacccgcgccgaagtcaaattcgaaggcgacaccctcgtcaaccgcatcgaactcaaaggcatcgacttcaaagaagacggcaacatcctcggacacaaactcgaatacaactacaacagccacaacgtctacatcatggccgacaaacaaaaaaacggcatcaaagccaacttcaaaatccgccacaacatcgaagacggcagcgtccaactcgccgaccactaccaacaaaacacccccatcggcgacggccccgtcctcctccccgacaaccactacctcagcacccaatccgccctcagcaaagaccccaacgaaaaacgcgaccacatggtcctcctcgaattcgtcaccgccgccggaatcacactcggcatggacgaactctacaaataaccacaacacaaccaacacaaaa

SEQ ID NO: 31 - DNA, 758 bp, Aequorea victoria
  eGFP C-depleted (maximum exchange of only C-containing but not U-containing codons)
gggatacgccgccaccatggtgagcaagggagaggagctgttcacaggggtggtgccaatcctggtcgagctggacggagacgtaaacggacacaagttcagcgtgtccggagagggagagggagatgcaacatacggaaagctgacactgaagttcatctgcacaacaggaaagctgccagtgccatggccaacactcgtgacaacactgacatacggagtgcagtgcttcagccggtacccagaccacatgaagcagcacgacttcttcaagtccgcaatgccagaaggatacgtccaggagcggacaatctccttcaaggacgacggaaactacaagacacgggcagaggtgaagttcgagggagacacactggtgaaccggatcgagctgaagggaatcgacttcaaggaggacggaaacatcctggggcacaagctggagtacaactacaacagccacaacgtctatatcatggcagacaagcagaagaacggaatcaaggcaaacttcaagatccggcacaacatcgaggacggaagcgtgcagctcgcagaccactaccagcagaacacaccaatcggagacggaccagtgctgctgccagacaaccactacctgagcacacagtccgcactgagcaaagacccaaacgagaagcgggatcacatggtcctgctggagttcgtgacagcagcagggatcactctcggaatggacgagctgtacaagtaaggagaagagaaggaagagaaaa

SEQ ID NO: 32 - DNA, 758 bp, Aequorea victoria
  eGFP C2-depleted (maximum exchange of all C-containing codons)
gggatacgccgccaccatggtgagcaagggagaggagctgttcacaggggtggtgccaatactggtggagctggacggagacgtaaacggacacaagttcagcgtgagtggagagggagagggagatgcaacatacggaaagctgacactgaagttcatatgcaccacaggaaagctgccagtgccatggccaacactggtgacaacactgacatacggagtgcagtgcttcagccggtacccagaccacatgaagcagcacgacttcttcaagagtgcaatgccagaaggatacgtgcaggagcggacaataagtttcaaggacgacggaaactacaagacacgggcagaggtgaagttcgagggagacacactggtgaaccggatagagctgaagggaatagacttcaaggaggacggaaacatactggggcacaagctggagtacaactacaacagccacaacgtgtatataatggcagacaagcagaagaacggaataaaggcaaacttcaagatacggcacaacatagaggacggaagcgtgcagctggcagaccactaccagcagaacacaccaataggagacggaccagtgctgctgccagacaaccactacctgagcacacagagtgcactgagcaaagacccaaacgagaagcgggatcacatggtgctgctggagttcgtgacagcagcagggataactctgggaatggacgagctgtacaagtaaggagaagagaaggaagagaaaa

SEQ ID NO: 33 - DNA, 615 bp, Mus musculus
  mEPO WT
gggaaactgccaagatgggggtgcccgaacgtcccaccctgctgcttttactctccttgctactgattcctctgggcctcccagtcctctgtgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacaggtgaggagaagagaaggaagagaaaa

SEQ ID NO: 34 - DNA, 615 bp, Mus musculus
  mEPO U-depleted (maximum exchange)
gggaaactgccaagatgggggtgcccgaacgacccaccctgctgctcttactctccctcctactgatccccctgggcctcccagtcctctgcgcacccccacgcctcatctgcgacagccgagtcctggagaggtacatcttagaggccaaggaggcagaaaacgtcacgatgggatgcgcagaaggacccagactgagcgaaaacatcacagtcccagacaccaaagtcaacttctacgcatggaaaagaatggaggtggaagaacaggccatagaagtctggcaaggcctgtccctgctctcagaagccatcctgcaggcccaggccctgctagccaactcctcccagccaccagagaccctccagctccacatagacaaagccatcagcggactacgaagcctcacatcactgctccgggtactgggagcacagaaggaactcatgtcgcccccagacaccaccccacccgcaccactccgaacactcacagtggacacattctgcaagctcttccgggtctacgccaacttcctccgggggaaactgaagctgtacacgggagaggtctgcaggagaggggacaggtaaccacaacacaaccaacacaaaa

SEQ ID NO: 35 - DNA, 615 bp, Mus musculus
  mEPO UC-depleted (maximum exchange)
gggaaactgccaagatgggggtgccagaacgaccaacactgctgctcctactcagcttgctactgatcccactggggctcccagtcctctgcgcaccaccaagactcatctgcgacagccgagtactggagaggtacatcctagaggcaaaggaggcagaaaacgtcacgatgggatgcgcagaaggaccaagactgagcgaaaacatcacagtcccagacacaaaagtcaacttctacgcatggaaaagaatggaggtggaagaacaggcaatagaagtatggcaagggctgagcctgctcagcgaagcaatcctgcaggcacaggcactgctagcaaacagcagccagccaccagagacactccagctccacatagacaaagcaatcagcggactacgaagcctcactagcctgctcagggtactgggagcacagaaggaattgatgtcgccaccagacacaacaccaccagcaccactccgaacactcacagtggacactttctgcaagctcttcagggtctacgcaaacttcctcagggggaaactgaagctgtacacgggagaggtctgcaggagaggggacaggtgaggagaagagaaggaagagaaaa

SEQ ID NO: 36 - DNA, 615 bp, Mus musculus
  mEPO UG-depleted (maximum exchange)
gggaaactgccaagatgggagtccccgaacgacccaccctcctcctcttactctccctcctactcatcccactcggcctcccagtcctctgcgcacccccacgcctcatctgcgacagccgagtcctcgaaagatacatcttagaagccaaagaagcagaaaacgtcacaatgggatgcgcagaaggacccagactcagcgaaaacatcacagtcccagacaccaaagtcaacttctacgcatggaaaagaatggaagtcgaagaacaagccatagaagtctggcaaggcctctccctcctctcagaagccatcctccaagcccaagccctcctagccaactcctcccaaccaccagaaaccctccaactccacatagacaaagccatcagcggactacgaagcctcacatcactcctccgcgtactcggagcacaaaaagaactcatgtcaccaccagacaccaccccaccagcaccactccgaacactcacagtcgacacattctgcaaactcttccgcgtctacgccaacttcctccgcggaaaactcaaactctacacaggagaagtctgcagaagaggagacagataaccacaacacaaccaacacaaaa

SEQ ID NO: 37 - DNA, 615 bp, Mus musculus
  mEPO C-depleted (maximum exchange of only C-containing but not U-containing codons)
gggaaactgccaagatgggggtgccagaacgtccaacactgctgcttttactctccttgctactgattcctctgggactcccagtcctctgtgctccaccacggctcatctgcgacagtcgagttctggagaggtacatcttagaggcaaaggaggcagaaaatgtcacgatgggttgtgcagaaggtccaagactgagtgaaaatattacagtcccagatacaaaagtcaacttctatgcttggaaaagaatggaggtggaagaacaggcaatagaagtttggcaaggactgtccctgctctcagaagcaatcctgcaggcacaggcactgctagcaaattcctcccagccaccagagacacttcagcttcatatagacaaagcaatcagtggtctacgtagcctcacttcactgcttcgggtactgggagctcagaaggaattgatgtcgcctccagatacaacaccacctgctccactccgaacactcacagtggatactttctgcaagctcttccgggtctacgcaaacttcctccgggggaaactgaagctgtacacgggagaggtctgcaggagaggggacaggtgaggagaagagaaggaagagaaaa

SEQ ID NO: 38 - DNA, 615 bp, Mus musculus
  mEPO C2-depleted (maximum exchange of all C-containing codons)
gggaaactgccaagatgggggtgccagaacgtccaacactgctgcttttactgagtttgctactgattcctctgggactgccagtgctgtgtgctccaccacggctgatatgcgacagtcgagttctggagaggtacatattagaggcaaaggaggcagaaaatgtgacgatgggttgtgcagaaggtccaagactgagtgaaaatattacagtgccagatacaaaagtgaacttctatgcttggaaaagaatggaggtggaagaacaggcaatagaagtttggcaaggactgagtctgctgagtgaagcaatactgcaggcacaggcactgctagcaaatagtagtcagccaccagagacacttcagcttcatatagacaaagcaataagtggtctacgtagcctgactagtctgcttcgggtactgggagctcagaaggaattgatgagtcctccagatacaacaccacctgctccactgcgaacactgacagtggatactttctgcaagctgttccgggtgtacgcaaacttcctgcgggggaaactgaagctgtacacgggagaggtgtgcaggagaggggacaggtgaggagaagagaaggaagagaaaa